(19) United States
(12) Patent Application Publication
(10) Pub. No.: US 2026/0053977 A1
(43) Pub. Date: Feb. 26, 2026

(54) EMBOLIC PARTICLES, METHODS OF FORMING NON-SPHERICAL SHAPED PARTICLES AND METHODS OF USING EMBOLIC PARTICLES TO OCCLUDE A TARGET VESSEL

(71) Applicant: Incept, LLC, Lexington, MA (US)

(72) Inventors: Amarpreet S. Sawhney, Lexington, MA (US); Hans Claesson, Lexington, MA (US); Raymond Lareau, Westford, MA (US); Benjamin Bell, Shrewsbury, MA (US)

(21) Appl. No.: 18/811,499

(22) Filed: Aug. 21, 2024

(51) Int. Cl. A61L 24/10; A61L 24/00

(52) U.S. Cl. CPC A61L 24/104; A61L 24/0042; A61L 2430/36

(57) ABSTRACT

Embolic particles of salted thermally crosslinked gelatin provide for temporary embolization for vessels within a patient. The embolic particles may be formed by drying a gel or liquid layer of gelatin and salt to form a dry salted gelatin sheet which can then be milled and sieved to a selected particle size range. The embolic particles may be characterized as a collection of particles with a majority of the particles having a platelet shape or ellipsoidal shape with a thickness or dimension no more than about 2000 microns. Useful sieved particle sizes include from 63 microns to 250 microns, or less than 63 microns. Methods of making the embolic particles from sheets of dry salted thermally crosslinked gelatin are described, as are methods for making the sheets. Methods and medical systems for performing temporary occlusion of a blood vessel are also described.

### FIELD OF THE INVENTION

The invention relates to embolic particles formed with thermally crosslinked gelatin to provide for temporary embolization of vessels within a patient. The invention further relates to methods of forming the embolic particles and methods of using the embolic particles for treatment of medical conditions through occlusion of blood vessels.

### BACKGROUND OF THE INVENTION

Emboli forming in the vasculature through blood clots can cause serious conditions, including myocardial infarction (heart attack), pulmonary embolic events, ischemic strokes and other problems. On the other hand, artificially introduced embolic particles can be used to cut off blood flow into locations to provide therapeutic efficacy. For example, bulges in vessels associated with some aneurysms can be embolized to stabilize the structure. Also, cutting off blood flow to some tumors or to sites of hemorrhage can lead to beneficial effects. Thus, embolic particles can be a desirable therapeutic tool, and many embolic particles are available for medical use.

For example, osteoarthritis (OA) is a common degenerative joint disease. It is characterized by pain and is generally accepted to be an inflammatory disease of synovial joints. Neovascularization can result from the chronic inflammation and contributes to further inflammation that may lead to pain and further degradation of the joint. Other orthopedic conditions associated with inflammation include, for example, back problems, and other joint conditions, such as frozen joints. Other conditions such as some forms of cancer or tumors have been treated with embolic particles. Occlusion of fine vasculature associated with inflammation or other medical conditions has been shown to provide therapeutic effects.

### SUMMARY OF THE INVENTION

In a first aspect, the invention pertains to a collection of dry gelatin particles. The dry gelatin particles may comprise crosslinked gelatin and have a sieve particle size of no more than about 2000 microns. A majority of the dry gelatin particles by weight may have a non-spheroidal shape with an aspect ratio of at least about 2. A majority of the dry gelatin particles by weight may have a platelet shape with an average thickness no more than about 2000 microns, from about 20 to about 2000 microns, from about 20 microns to about 1000 microns, from about 20 microns to about 500 microns, or from about 25 microns to about 200 microns. Dry gelatin particles having a platelet shape may have a thickness variation no more than about +50 microns. The average thickness of a collection averages across the expanse of each platelet and averages over all of the platelets of the collection. Dry gelatin particles having a platelet shape may comprise a plate having a perimeter that is irregular with respect to individual platelets and between platelets of the collection. The collection of dry gelatin particles may have different sieve particle sizes and/or sieve particle size distributions. The sieve particle size may be from 63 microns to 250 microns, or less than 63 microns. The sieve particle size distribution may be from about 20 microns to about 1000 microns, or from about 63 microns to about 250 microns.

The dry gelatin particles may comprise a metal halide salt. The dry gelatin particles may comprise from about 1 wt % to about 66 wt % of the metal halide salt, for example, from about 2 wt % to about 40 wt %, from about 1.5 wt % to about 55 wt %, or from about 2 wt % to about 40 wt %. The metal halide salt may comprise, for example, sodium chloride, calcium chloride or mixture thereof.

The dry gelatin particles may comprise crosslinked gelatin. The dry gelatin particles may comprise thermally crosslinked gelatin whereby crosslinking was carried out at a temperature from about 100° C. to about 200° C. for from about 5 minutes to about 48 hours. The dry gelatin particles may be formed by crosslinking gelatin dried from a gelled form or a melted state. The dry gelatin particles may be formed from gelatin having an uncrosslinked bloom value from about 50 to about 325. Upon hydration, the hydrated gelatin particles may have a swelled density providing suspension in a 50:50 by weight blend of saline and contrast agent having a density of about 1.20 g/mL, which suggested a density of the swelled particles greater than this value.

The gelatin particles may be characterized by in vitro pepsin digestion and/or an in vivo recanalization rate. For example, the collection of gelatin particles may have an in vitro pepsin digestion time of from about 2 minutes to about 48 hours or from about 5 minutes to about 24 hours. For another example, the gelatin particles may have an in vivo recanalization rate of from about 30 minutes to about 60 days.

In another aspect, the invention pertains to a method for forming biocompatible gelatin particles. In some embodiments, the method may comprise pouring a warm solution of dissolved gelatin and salt into a tray followed by drying to form a dried gelatin sheet. In alternative embodiments, a dried gelatin sheet may be formed by drying gelatin foam imbibed with an aqueous salt solution. The salt may comprise a metal halide salt. The metal halide salt may be present within a range of amounts such that the dried gelatin sheet comprises from about 1 wt % to about 66 wt %, from about 2 wt % to about 40 wt %, from about 1.5 wt % to about 55 wt %, or from about 2 wt % to about 40 wt % of the metal halide salt. The metal halide salt may comprise, for example, sodium chloride, calcium chloride or mixture thereof. The dried gelatin sheet may have an average dry thickness no more than about 2000 microns, from about 20 microns to about 2000 microns, from about 20 microns to about 1000 microns, from about 20 microns to about 500 microns, or from about 25 microns to about 200 microns.

The method may comprise drying the solution of dissolved gelatin and salt under certain conditions. For example, prior to drying, the solution may be allowed to gel or remain as a solution. For either option, drying may be carried out at a temperature above or below the melting point of the gelatin. For example, if the gelatin was allowed to gel, drying may be at a temperature below the melting point of the gelatin. After drying, the gelatin of the dried gelatin sheet may be crosslinked as described above.

The method may include breaking up the dried gelatin sheet into fragments, followed by grinding or milling to form the biocompatible gelatin particles as ground or milled dry gelatin particles. Grinding or milling can be carried out using a mill at a milling rate from 1000 rpm to about 30,000 rpm for a time from about 15 seconds to about 40 minutes. The method may further comprise sieving the milled dry gelatin particles by passing the particles through a mechanical sieve that vibrates the particles over a selected sieve mesh. The milled dry gelatin particles may be sieved such that the collection of particles has a sieve particle size within a particular range and/or sieve particle size distribution as described above.

In another aspect, the present invention pertains to a method for making a sheet of salted thermally crosslinked gelatin. The sheet of salted thermally crosslinked gelatin may include gelatin and salt. The method may comprise pouring a warm solution of dissolved gelatin and salt into a tray followed by drying to form a sheet of the dry salted thermally crosslinked gelatin. The sheet may be formed by drying gelatin foam imbibed with an aqueous salt solution. The salt may comprise a metal halide salt. The metal halide salt may be present in a dried form of the sheet at an amount ranging from about 1 wt % to about 66 wt %, from about 2 wt % to about 40 wt %, from about 1.5 wt % to about 55 wt %, or from about 2 wt % to about 40 wt %. The metal halide salt may comprise, for example, sodium chloride, calcium chloride or mixture thereof. The sheet of salted thermally crosslinked gelatin may have an average dry thickness no more than about 2000 microns, from about 20 microns to about 2000 microns, from about 20 microns to about 1000 microns, from about 20 microns to about 500 microns, or from about 25 microns to about 200 microns. The dry salted thermally crosslinked gelatin sheet may comprise from about 1.5 wt % salt to about 50 wt % salt and have an initial dry average thickness from about 25 microns to about 300 microns. The method for making the sheet of salted thermally crosslinked gelatin may comprise drying the solution of dissolved gelatin and salt under certain conditions as described above. After drying, thermal crosslinking of the dried gelatin sheet may be carried out as described above. The resulting sheet of dry salted thermally crosslinked gelatin may be broken up, milled, and sieved as described above.

In another aspect, the present invention pertains to a method for performing temporary occlusion of a blood vessel. The method may comprise injecting hydrated crosslinked gelatin particles described herein into the parent vessel of target vasculature for occlusion. The target vasculature may comprise fine vasculature associated with inflammation accompanying neovascularization, and neovascularization may be related to osteoarthritis.

Any of the salted crosslinked gelatin particles described herein may be hydrated and employed in the method for performing temporary occlusion of a blood vessel. For example, the hydrated salted crosslinked gelatin particles may be a hydrated form of salted thermally crosslinked gelatin particles described herein. For example, the hydrated salted crosslinked gelatin particles by weight may have an ellipsoidal shape with an aspect ratio of at least about 3, wherein the dry particles prior to hydration have a sieved particle size of no more than about 2000 microns and wherein the target vessel completely occludes and recanalization of the vessel occurs in a period of time from about 30 minutes to about 60 days or from about 10 minutes to about 60 days.

The method for performing temporary occlusion of a blood vessel may comprise injecting a solution or dispersion of the hydrated crosslinked gelatin particles. The solution or dispersion may comprise contrast agent and aqueous saline, such as contrast agent and roughly 50 percent by volume of saline. The amount of injected solution or dispersion may be any amount, for example, a volume from about 0.5 ml to about 10 ml, with particles present in any amount such as from about 10 mg to about 400 mg.

In a further aspect, the present invention pertains to a medical system for delivery of embolic material. The medical system may comprise a catheter and a delivery component. The catheter may comprise a hub and a tubular element, with the catheter being suitable for delivery through a patient's vasculature to reach a target vessel or the vicinity thereof. The delivery component may comprise a reservoir with a connector and an engagement element to deliver material from the delivery component through the connector. The connector may be suitable to connect to the hub of the catheter to form a sealed engagement in a configuration to deliver the embolic material through the catheter and out an end of the tubular element. The embolic material may comprise any of the particles described herein, for example, any of the salted crosslinked gelatin particles described herein, and which are in a biocompatible suspension fluid. The reservoir of the delivery component may be configured to comprise the embolic material having a volume from about 0.5 ml to about 10 ml, with particles present in any amount such as from about 10 mg to about 400 mg. The embolic material may comprise contrast agent and aqueous saline, such as contrast agent and roughly 50 percent by volume of saline.

### BRIEF DESCRIPTION OF THE DRAWINGS

FIG. 1 is a fragmentary side view of a catheter system suitable for the delivery of embolic particles into the vasculature.

FIG. 2A is a fragmentary view of the distal end of a catheter within a patient's vasculature showing delivery of embolic particles into a target branch artery to form an embolism.

FIG. 2B is a fragmentary view of the distal end of a catheter within a patient's vasculature showing delivery of embolic particles into a target parent artery to form an embolism.

FIG. 3A depicts a general flow procedure for forming embolic particles from salted gelatin according to an embodiment of the present invention.

FIG. 3B depicts a general flow procedure for forming embolic particles from salted foamed gelatin according to another embodiment of the present invention.

FIG. 4A shows the flow model used to evaluate performance of embolic materials.

FIG. 4B is a schematic diagram of the vascular model portion of the flow model shown in FIG. 4A.

FIG. 5 is an optical micrograph of dry gelatin particles prepared from a salted gelatin sheet having a thickness of about 50 μm and which are ground and sieved to a size range of 63-250 μm.

FIG. 6 is an optical micrograph of dry gelatin particles prepared from a salted gelatin sheet having a thickness of about 150 μm and which are ground and sieved to a size range of 63-250 μm.

FIG. 7 is an optical micrograph of dry gelatin particles prepared from a salted gelatin sheet having a thickness of about 50 μm and which are ground and sieved to a size range of less than about 63 μm.

FIG. 8 is a bar graph showing dissolution times for gelatin/salt particles either not thermally conditioned or only slightly thermally conditioned.

FIG. 9 is an optical micrograph of dry gelatin particles prepared from a gelatin sheet (no added salt) having a thickness of about 100 μm and which are ground and sieved to a size range of 63-250 μm.

FIG. 10 is an optical micrograph of dry gelatin particles prepared from a gelatin sheet (no added salt) ground and sieved to a size range of less than about 63 μm.

FIGS. 11A-11E are optical micrographs of dry gelatin/salt particles prepared from salted gelatin sheets having salt concentrations of 5.09 wt %, 9.64 wt %, 17.4 wt %, 29.2 wt % and 44.1 wt %, respectfully, and sieved to a size range of 63-250 μm.

FIG. 12 is a schematic cross sectional view of a testing apparatus used to evaluate packing and leaking of embolic materials.

FIGS. 13A and 13B are images at 75× and 100× magnifications, respectively, of dry gelatin/salt particles prepared as described for FIG. 6. The images were obtained using scanning electron microscopy (SEM).

FIGS. 14A-14C are images at 75×, 90×, and 250× magnifications, respectively, of dry gelatin/salt particles prepared as described for FIG. 6. The images were obtained using SEM.

FIGS. 15A-15D are images at 100× magnification of hydrated gelatin/salt particles prepared as described for FIG. 6. The images were obtained using SEM.

FIGS. 16A and 16B are optical micrographs of shredded and ground gelatin foam.

FIG. 17 is an optical micrograph of salted ground gelatin foam particles sieved to a size range of 63-250 μm.

### DETAILED DESCRIPTION OF THE INVENTION

Most embolic particles are available in the form of spherical particles made from resin, natural or synthetic hydrogels and acrylic polymers. These spherical particles are often of a tight size dispersion and the practitioner must choose the right size for a particular therapeutic application. In order to embolize fine blood vessels, such as neovasculature, while preserving the larger vessels feeding them or neighboring them, treating with spherical beads can lead to larger zones of vascular occlusion and attendant undesirable side effects of loss of sensation, skin discoloration, ulceration etc. Also, these spherical particles may not pack well and sometimes fail to generate reproducible vascular occlusion due to gaps in the packing of these particles. Irregular shaped particles have been used from Gelfoam powder or by chopping up pieces of Gelfoam sheet, but do not possess a predictable physiological absorption and may have too long an absorption time for embolization in musculo-skeletal disease treatment applications.

Non-spherically shaped gelatin particles, such as platelet shaped particles in dry form, have been developed that are suitable for use as embolics, with appropriate sizes and digestion/dissolution properties for temporarily occluding target blood vessels with a selected absorption time to allow for recanalization after an appropriate occlusion time to provide a desired physiological outcome, such as atrophying excessive microvasculature formed through undesirable angiogenesis caused by cancer or inflammation. Absorption time can be selected also to reduce consequences of off target embolization. Dry refers to non-hydrated particles. As described in the examples, the dried particles are formed from drying gelation solutions. Gelatin is hydrophilic, so it absorbs a small amount of atmospheric moisture. As a result, “dry” particles are generally in equilibrium with atmospheric moisture, which is not significant in the context of particle properties and ultimate hydration for use. The particles generally have shapes that are non-spheroidal such that they can orient in a flow and pack to form a tight leak resistant occlusion. Additionally, the higher surface to volume ratio afforded by the non-spherical nature, allows the particles to remain suspended for longer duration in contrast media used for interventional applications. In embodiments of particular interest, the dry particles can be primarily platelet shaped, and the hydrated particles can be described as generally ellipsoidal in overall appearance. Gently thermally crosslinked gelatin is a desirable material for this purpose due to formation of a faster absorbing embolic particle. The non-spherical shape of the particles allows for a more complete packing of the embolic and thus generating a more complete occlusion compared to spherical beads. In contrast with the current non-spherically shaped particles, spherical particles are demonstrated to have some leaking after the spherical embolic particles are delivered. In some embodiments, the gelatin particles can be produced by forming a dry layer of thermally crosslinked gelatin, with added salt, and grinding the dry layer of gelatin to form the particles. Desirable results can be obtained using essentially dense sheets of dry gelatin. The gelatin can be thermally crosslinked in the sheet form prior to grinding. In alternative embodiments, foamed gelatin sheets can be salted and ground to form the particles. The grinding process results in the formation of non-spherically shaped particles, which are primarily platelet shaped when dry with an aspect ratio that can be defined as the ratio of the largest diameter divided by the thickness. The non-spherical shape of the gelatin particles provides for desirable particle packing when delivered for occlusion. Salt is added to the gelatin to improve the process conditions since the salt makes the gel more brittle so that it breaks easier into the desired irregularly shaped particles upon grinding. The salt also makes the gelatin easier to process by diffusing any static charge built up during the grinding process. The process improvement includes increased brittleness, improving grinding yield, reduce static build up, improving sieving and handling. In addition, the presence of salt also improves the hydrophilicity which results in a faster suspension in contrast agent. At the same time, the salt also speeds hydration of the gelatin particles due to diffusion driven by osmotic pressure. Thermal crosslinking can be used to stabilize the particles from rapid dissolution in physiological solution while allowing the particles to degrade in vivo in an appropriate period of time. The degree of crosslinking can be used to control the degradation time in vivo, which can range from short periods of time to relatively long periods of time.

The processing described herein is directed to forming non-spheroidal shaped particles, which is intended to imply that the particles do not have substantially curved surfaces in dry form, although the hydrated particles have softened edges and expansion that forms shapes that can be generally referred to as ellipsoidal, although in a general and not mathematical sense. The processing can result in variability between the particles. Sieving can reduce the variability, but significant variability can remain. Nevertheless, significant numbers of appropriately sized particles can have similar shapes. To characterize the particle shapes in a meaningful way, reference can be made to a majority of the particles by weight, such that very small particles that can have different shapes, cannot influence the evaluation significantly, and generally the particles can have sufficient uniformity that characterization can be performed empirically without the need for a rigorous systematic measurement.

In this context, non-spheroidal is not intended to imply anything with respect to a mathematical sense, but in a qualitative sense usual for polymer articles, evaluated visually. The particles can also be characterized with respect to an aspect ratio of at least about a factor of 2, or at least about 3, as explained in more detail below. The thickness of the sheets ground to make the particles can correspond to a dimension of the particles, such as the smallest dimension in the context of an aspect ratio. The thickness values are explained below to be not precisely fixed due to variability in the thickness. As noted above, the majority (by weight) of the dry particles can be described as platelet shaped, and the majority of the hydrated particles can be described as ellipsoidal in shape. In either dry or hydrated form, the particles generally can be characterized along three axes, with a thickness, a length (generally along the longest dimension), and a third dimension generally picked to correspond to the, possibly orthogonal, size that suggests the overall particle size. Overall, the qualitative shapes of the particles can be described with appropriate certainty through the characterization as platelet (dry)/ellipsoidal (wet) and having an aspect ratio of at least two for a majority of the particle by weight. The aspect ratio can be specified as the longest axis dimension across the particle (generally through the particle center) divided by the shortest axis. Visualization of the particles in optical micrographs can provide this information readily for most particle collections since a large number of the particles in the center of the particle distribution can be easily identified as having these properties.

The platelet/ellipsoid shaped gelatin articles can be used to occlude selected blood vessels. Generally, the particles are released in a parent blood vessel leading to a target vessel where the particle flow and occlude the target vessel until the particles are absorbed and flow is restored. Embolic particles can be effectively used for reducing or eliminating blood flow to neovasculature developed due to the inflammatory disease process associated with acute or chronic inflammation. Although the embolic particles can be used for any reasonable medical application where temporary occlusion is desirable. This can for example be useful for increasing the residence time of chemotherapeutic drugs being delivered into tumors. The temporary embolic particles can have attendant dissolved or suspended drug in the aqueous phase of the embolic suspension. When this drug and embolic mixture is delivered within the tumors, it can occlude the blood flow while retaining the drug within the tumor for a period of time. Absorption of the embolic in a short duration (hours to days) allows for recanalization and re-access of the tumor bed, should it be needed in the future.

Embolic particles have been used for some time for treatment of certain cancers, in particular liver cancer, to cut off blood supply. Use of embolic particles for treatment of solid tumors in such organs as the liver is well established and is referred to as transcatheter arterial chemoembolization (TACE) with an eluting chemotherapy drug or transarterial bland embolization (TAE) without an eluting drug. Commercial embolic particles are available for temporary or permanent occlusion of blood vessels, such as Embozene® microspheres (Varian, long term occlusion) and Embosphere® microspheres (Merit Medical, permanent). Gelatin based microspheres for use as embolic particles (GelBead™ from Teleflex) are described in Japanese patent 4422191 B1 to the Jellice Company (hereinafter the '191 patent), which are described further below.

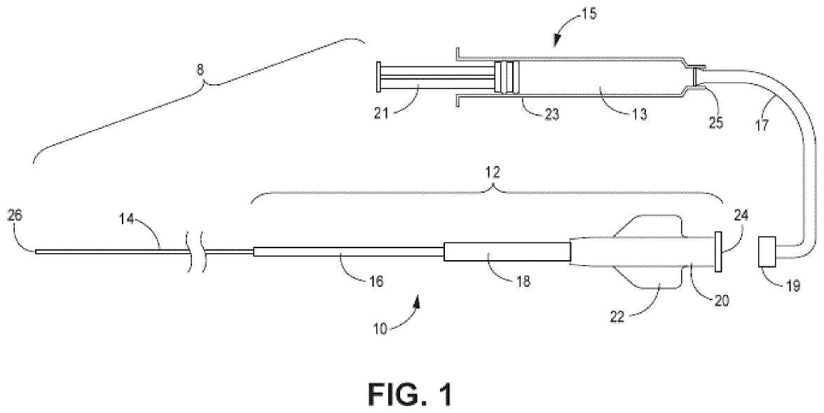

Embolic particles are generally delivered into the vasculature using a catheter to access a desired location for particle delivery with a syringe or the like used to deliver a suspension of the particles through the catheter. FIG. 1 depicts a catheter system 8 comprising catheter 10 having hub 12 and tubular element 14. Hub 12 has intermediate portion 16, strain relief member 18, hub wings 22 and proximal hub connecter 24. Tubular element 14 has distal outlet tip 26. Strain relief member 18 provides a transition from flexible shaft 14 to hub 20. Intermediate portion 16 is optional and may be provided as a further strain relief member over shaft 14 and/or as a portion of shaft 14 that has a large inner diameter (ID) and/or outer diameter (OD). Referring to FIG. 1, catheter system 8 also comprises embolic particles delivery components having a reservoir 13 with a suspension of embolic particles and a delivery device 15, such as a syringe or a pump, flow tubing or the like 17 and a generally a connector 19, such as a luer fitting for attachment to proximal hub connector 24. As shown in FIG. 1, delivery device 15 is a syringe with plunger 21, barrel 23 and connector 25 for attachment of flow tubing 17. Suitable catheters are commercially available from a variety of sources. Artisans are familiar with these components and their operation, as well as their introduction and use in cooperation with guide wires, hemostatic introducers and other components for catheter procedures. The vasculature can be accessed through an appropriate vessel depending on the target location. Suitable vessels for accessing the vasculature include the femoral artery in the leg or radial artery in the wrist or arm.

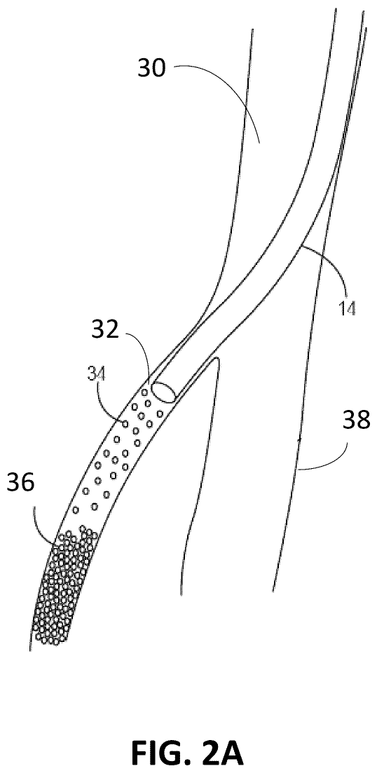

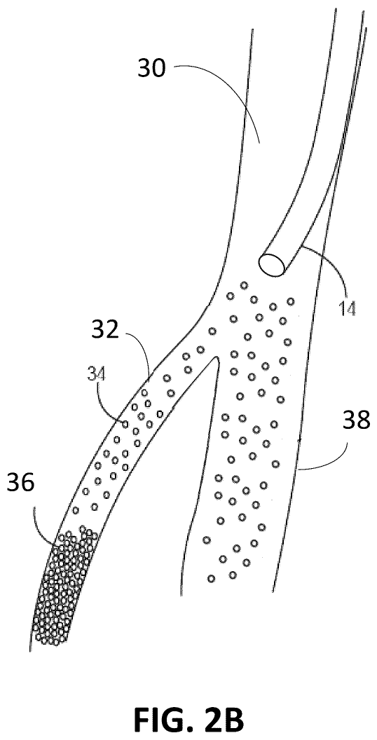

Delivery of the particles in exemplary catheter based procedures are depicted in FIGS. 2A and 2B. The catheter is introduced using hemostatic procedures, such as those generally used in the art. Parent artery 30 branches into first branch artery 32 and second branch artery 38 having a diameter between that of the parent artery and the first branch artery. Referring to FIG. 2A, catheter shaft 14 is introduced through parent artery 30 and positioned at a target vasculature within first branch artery 32. An embolizing material, for example embolic particles 34, is injected through catheter shaft 14. Embolic particles 34 bridge across first branch artery 32 to form embolism 36 that blocks blood flow. After delivery of the embolic particles, catheter shaft 14 is withdrawn. Referring to FIG. 2B, catheter shaft 14 is introduced into parent artery 30, such that upon release of embolic particles 34, the particles flow into both first branch artery 32 and second branch artery 38. In this particular example, embolic particles 34 bridge across first branch artery 32 but not second branch artery 38, and embolism 36 forms only in first branch artery 32.

Gelatin is a hydrolysis product of collagen, a major component of the extracellular matrix in animals. Collagen is generally harvested from various livestock. Collagen is an insoluble fibrous protein in its native form. The hydrolysis breaks or denatures the protein polymer strands into smaller units such that the resulting gelatin can be processed. The precise nature of gelatin can depend on the processing. But generally, the gelatin is soluble in warm/hot water and some other polar solvents. To revert the gelatin to be insoluble, the gelatin can be crosslinked to control the specific properties. To avoid the use of toxic chemicals for crosslinking, sufficient crosslinking can be achieved with heat to achieve thermal crosslinking. Heat crosslinking is also more desirable when a more rapidly absorbed gelatin particle formulation is desired. While thermal crosslinking is desirable, some chemical crosslinking can be used in some embodiments. Thermal crosslinking involves bonding between peptide functional groups, while chemical crosslinking involves a chemical crosslinking agent that bridges between peptide functional groups. A high degree of chemical crosslinking can render the structure stable against breakdown, and chemical crosslinking has been used to prepare prosthetic tissue (a collagenous material) for long term implantation into patients. Due to chemical differences between thermal crosslinking and chemical crosslinking, analytical study can be used to evaluate processing technique if desired. For example, chemically crosslinked gelatin and especially gelatin foams are often crosslinked with glutaraldehyde or formaldehyde, which can induce more permanent crosslinks, thus making the materials less suitable for shorter term embolization applications where more rapid absorption of the gelatin material may be desirable. Radiation driven crosslinking can also crosslink the gelatin, with variable levels of crosslinking introduced by levels of radiation delivered.

Gelatin can also contain endotoxins from bacteria which can be carried by the harvested collagen, so sufficient purification should be used to reduce the endotoxin levels. Commercially obtained gelatin can be appropriately purified to reduce endotoxins to medical grade levels. In addition to endotoxin levels, commercial gelatin can be characterized by molecular weight, which is determined by the degree of hydrolysis of the collagen. Alternatively, the gelatin can be characterized by a Bloom number, which is a measure of the mechanical strength of the gelatin. The Bloom number can be correlated with the molecular weight of the gelatin.

The '191 patent, cited above, describes forming thermally crosslinked gelatin particles for use as embolic particles. To form the particles from a solution of gelatin in the process of the '191 patent, the solution is spray dried. The spray drying process results in particles that are substantially spherical and with a broad size distribution. In the '191 patent, the gelatin particles are thermally crosslinked, and the processing starts with gelatin powder with a low endotoxin level. The '191 patent describes crosslinking for a time of 1 hour to 168 hours at a temperature from 100° C. to 160° C., although specific embodiments are described for heating for 24 hours at 110° C. to 150° C. The '191 patent does not characterize their particles with respect to density or porosity, although spray drying conditions can influence these properties. Relative to the Jellice embolic particles, the embolic particles described herein have desirable non-spherical, e.g., platelet (dry) or ellipsoidal (wet), shapes and good flow/handling properties that facilitate their manufacture and use. Plate shaped particles when dry can be regular in the sense of having a common thickness for a majority of the particles but can be considered irregular in some sense relative to spheroidal particles since the ellipsoidal particles herein generally have a broader distribution of sizes and shapes due to process approach relative to methods used to make spheroidal particles. The higher surface to volume ratio of these non-spherical particles allows for them to remain suspended within contrast agents used for interventional procedures. Thus, relative to spherical particles, constant agitation and re-suspension of the hydrated ellipsoidal shaped particles is not needed. Also, these particles hydrate sooner due to the presence of salt in their formulation.

Neovascularization in hypervascular vessels can result from chronic inflammation and can contribute to further inflammation that may lead to pain and further degradation of the joint. Okuno et al. (hereinafter Okuno 2017 article), “Midterm Clinical Outcomes and MR Imaging Changes after Transcatheter Arterial Embolization as a Treatment for Mild to Moderate Radiographic Knee Osteoarthritis Resistant to Conservative Treatment,” J Vasc Interv Radiol (2017) 28:995-1002, incorporated herein by reference. Applicant previously described temporary embolization to occlude vessels for the treatment of undesirable neovascularization accompanying hypervascular vessels associated with joint inflammation. See, published U.S. patent application 2020/0368402 to Sawhney et al. (hereinafter the '402 application), entitled “Embolization With Transient Materials,” incorporated herein by reference. The use of gelatin particles was described in the '402 application but only starch embolic particles were exemplified. The gelatin particles described herein provide improved handling, product consistency and desirable performance in use compared with the previous starch particles.

Okuno and coworkers have conducted studies of embolization of blood vessels associated with joint inflammation. Okuno et al. reported results of experiments using embolization of neovascular vessels of arteries with 75-μm polymer microspheres (EMBOZENE®) or with Imipenem/cilastatin sodium (IPM/CS; PRIMAXIN; Merck, Whitehouse Station, New Jersey) in iodinated contrast medium (HEXABRIX; Terumo, Tokyo, Japan). See Okuno 2017 article cited above. IPM/CS is reported to be a crystalline compound that is slightly soluble in water and forms particles with an embolic effect when it is suspended in contrast medium. More recent work has extended this earlier study. Okuno et al., “Transarterial Embolization of Neovascularity for Refractory Nighttime Shoulder Pain: A Multicenter, Open-Label, Feasibility Trial,” Journal of Vascular and Interventional Radiology, Vol. 33 (12), December 2022, Pages 1468-1475, incorporated herein by reference. While the IPM/CS particles are transient, they are an antibiotic and not designed or approved specifically for embolic use and are not desirable for applications where there is no infection present as overuse in this manner can contribute to antibiotic resistance. Thus, it is desirable to have an embolic particle that does not by itself have medicinal properties that are not relevant the medical condition being treated.

In some embodiments, gelatin is the starting point of the synthesis of the plate shaped embolic particles described herein. The drying techniques are designed to achieve good particle density, and a desired degree of heating can achieve a selected degree of thermal crosslinking under selected conditions. In particular, the gelatin can be formed into sheets with a selected thickness that corresponds to a thickness dimension of the ultimate particles. The gelatin sheets are made by pouring a gelatin containing solution into a tray. The gelatin containing solution may be allowed to cool to form a gel. Drying can be performed at a sufficiently low temperature to avoid melting the gel, although the drying can be performed at a higher temperature at which the gel is melted. Drying the gelatin in a gel form is believed to help orient the gelatin to improve crosslinking, although suitable particles may be formed through thermal crosslinking of gelatin dried from a melted state. Air or other gas, such as nitrogen, can be blown across the trays to facilitate drying. The drying may be performed with little or no heat. After drying, the gelatin has formed sheets, and the sheets are ground to form the particles. The ground particles can be sieved to eliminate small particulates and any larger chunks of gelatin.

In other embodiments, the particles can be prepared from gelatin foam sheets. The gelatin foam is soaked in a salt solution, and the process for salting the foamed gelatin sheets is different from salting the gelatin for the dense gelatin sheets, as described below. Foamed gelatin sheets can be obtained commercially. The gelatin foam or sponges are believed to be produced with some form of crosslinking. Since the processes are proprietary, it is generally not known how the crosslinking is performed and may or may not be chemically crosslinked with toxic crosslinkers such as glutaraldehyde. Use of non-chemically crosslinked gelatin avoids concerns about release of potentially toxic chemicals used for crosslinking and allows control of the digestion or absorption times according to thermal crosslinking conditions. The foamed gelatin material generally has a porous structure with a sponge-like cell structure, and the porous structure allows for introducing the salt into the foamed sheet. Gelatin foam sheets may or may not be further thermally crosslinked either before and/or after salting. Commercial foamed gelatin may be distributed in blocks that are cut to a desired thickness. The formation of a gelatin sponge is known, which may be stabilized with chemical crosslinking. See, for example, published U.S. patent application 2007/0077274 to Ahlers (the '274 application), entitled “Method for Producing Shaped Bodies Based on Crosslinked Gelatin,” incorporated herein by reference. The '274 patent refers to previous crosslinked gelatin products that have insufficient persistence for certain applications. The crosslinked gelatin of the '274 patent is formed with a pore forming agent, such as air to provide for the sponge structure with pores. The products in the '274 patent are still absorbable. Gelatin sponges are commercially available for use as hemostatic materials. For example, gelatin sponges are available from Gelita AG (Germany, applicant of the '274 patent) and Ethicon (U.S.). Gelita and Ethicon sell gelatin sponges as hemostatic patches, Surgi-Foam® (Ethicon) and Gelita-Spon® (Gelita). Non chemically crosslinked gelatin sponges are desired for this application.

Salt added to the gelatin significantly changes the mechanical properties in the gelatin sheets that contributes to the desirable particle formation and also provides significant property improvements to the product particles. Depending on the particle production approach (dense gelatin sheet versus foamed gelatin sheet), salt is added using different processing, as described further below. In particular, the salted gelatin in sheet or particulate form is more brittle, appear to have reduced static charge. As a result, the sheets grind to fragment into the desired irregular, such as plate, shaped particles with a good yield. Also, the particulates exhibit significantly improved flow properties. The shape of the particles facilitates good packing of the particles such that effective and rapid occlusion can occur with a lower mass of particles. The improved flow properties allow for easier sieving, and the particles are significantly easier to transfer into vials for packaging. When forming dense gelatin sheets, salt is added to the gelatin solution prior to drying and can be dissolved in the solution before adding the gelatin. When using foamed gelatin sheets, a salt solution can be infused into the foam for salting.

The ellipsoidal shaped hydrated particles have a larger surface area to volume ratio relative to more spherical particles. The increased relative surface area promotes suspension of the particles in a liquid with reduced settling. Due to the improved ability to suspend the particles they can remain suspended better in contrast solution or alternative delivery fluid. Thus, due to reduced agglomeration of the particles to each other, the salted gelatin particles provide improved injectability relative to irregular foam particles, such as Gelfoam or spherical foam particles made from polyvinyl alcohol (PVA). Also, the plate shaped salted gelatin particles provide better suspension and packing upon delivery relative to spherical particles. Generally, as the amount of salt is increased the processing continues to improve up to a limit beyond which the gelatin can become too brittle.

With appropriate processing parameters, the ground gelatin particles can be substantially plate shaped for a majority of the dry particles with one smaller thickness dimension relative to the orthogonal direction defining the planar expanse of the plate. This shape of the particles provides desirable features. The aspect ratio has a relatively broad distribution due to the irregularities of the particle shapes, but that is an appropriate feature of the particles. The thickness of the plates can be determined by the thickness of the layer prior to grinding. The aggressiveness of the grinding process influences the other dimensions. With appropriate grinding, few small particles are created and collected as waste from a sieving process. The grinding can be performed using various commercial grinders, such as a knife grinder or a rotor mill, as described further below.

The improved gelatin particle characteristics can be suitable for a range of medical applications. While the basic particle properties, such as the salted gelatin composition and plate shape, are suitable for the range of applications, two parameters that can be selected specific for certain applications are the particle sizes and the digestion rate. The embolic particles can be characterized by an in vitro enzymatic digestion test, which is expected to correlate with trends for in vivo absorption rate and recanalization rates, although the in vitro tests are not designed for quantitative comparisons. Actual observed recanalization rates generally depend on the specifics of the particular blood vessel. The particle sizes can be selected through a sieving process, as described further below. Also, the particle size distribution is influenced by the grinding process, as well as the thickness of the dried gelatin films for the dense gelatin sheet embodiments and the characteristics of the foamed gelatin for these embodiments. The absorption rate can be adjusted using the particle size and the crosslinking process. The selection of the absorption rate can be influenced by the target application of the particles. In some embodiments, embolic particles can be used for treatment of hypervascular tumors, such as liver tumors (hepatoma), uterine fibroids, and the like, arteriovenous malformations, prostatic artery embolization, hemostasis of arterial bleeding, symptomatic benign prostatic hyperplasia, and hypervascular vessels associated with joint inflammation which may result from chronic disease or from acute injury, as well as other indications that may be suitable for embolic particle treatment.

### Gelatin Particle Characterization

The gelatin particles in dry form may have a plate shape with the shape and size influenced by the process conditions, which can be referred to as platelets. The resulting particles can be characterized by composition, size, size distribution, density and dissolution time. These properties may depend on several process parameters, as explained further below. Due to the salting of the gelatin, the particles exhibit very low levels of agglomeration, good sieving properties, easy delivery from a catheter, rapid hydration, and good packing in a vessel upon delivery into a patient. By avoiding agglomeration during the sieving process, the sieving process better results in appropriately characterized particle collections following the sieving process. The improved sieving thus provides consistent, good embolic performance.

While the dry particles are generally platelet shaped, the particles exhibit a range of particle sizes so there may not be a single qualitative descriptor that describes all of the particles, and this depends somewhat on the thickness relative to the sieve sizes and the grinding. Generally, but not necessarily, for the dried dense gelatin sheet embodiments, the ground particles have a dimension corresponding to the thickness of the sheet of gelatin that is ground into the particles. The particles are generally irregularly shaped and the grinding process can fragment the particles along the plate thickness for at least some of the particles. For particles ground from dense sheets, the thickness of the sheets provides a limit on one dimension of the particles since the grinding does not generally result in formation of hard particle agglomerates fusing intermediate particles together. The plate shaped particles usually have a plate surface resulting from the grinding with relatively planar surfaces spaced apart by the sheet thickness. In any case, the particles are angular and are not spherical, ovoid or similarly rounded structures. For simplicity in reference to these particle shapes, these particles are described as non-spheroidal, which is intended to summarize the concepts of the previous sentence and not to refer to any mathematically precise meaning. Upon hydration, the particles remain non-spheroidal, but the angular nature is softened, so the particles can be referred to as ellipsoidal in a qualitative and non-mathematical sense.

Also, the particles can be characterized by an aspect ratio to identify the deviations from cubic types forms and corresponding particles that fill a large fraction of a cube, such as a sphere. Larger aspect ratio particle shapes, such as tile shapes, are found to provide good packing to form leak resistant embolic particles. Particles can be characterized along principal axes based on the moments of inertia or from a practical sense with an axis along the longest distance between two points along the surface and two axes in the plane through the center of the particle perpendicular to the axis along the longest distance. For estimates, these can be evaluated visually. Generally, the processing results in roughly plate shaped particles, with two relatively flat surfaces, and these are referred to as platelets, although not wanting to imply a direct relationship in shape or otherwise with platelet blood components In some embodiments, the thickness of the sheets ground to make the particles can correspond to a dimension of the particles, such as the smallest dimension in the context of an aspect ratio. The thickness values are explained below to be not precisely fixed due to variability in the thickness. Generally, the particles, such as platelet shaped particles, can have an aspect ratio of at least about 2, and the aspect ratio generally follows from the processing parameters. The aspect ratio refers to the ratio of the longest distance between two particle surface points and the shortest orthogonal distance, which may or may not relate to a platelet shape with two longer dimensions. In further embodiments, the aspect ratios can be at least about 2.5, and in other embodiments from about 3 to about 20. The platelet shaped particles can have two larger dimensions relative to a thickness, or a shard shape with one larger dimension and two smaller dimensions. A person of ordinary skill in the art will recognize that additional ranges of aspect ratio within the explicit ratios above are contemplated and are within the present disclosure.

To characterize the particle shapes in a meaningful way, reference can be made to a majority of the particles by weight, such that very small particles that can have different shapes, do not influence the evaluation significantly, and generally the particles can have sufficient uniformity that characterization can be performed empirically without the need for a rigorous systematic measurement. Thus, particle collections can be characterized in dry form by a majority of the particles by weight having indicated shape parameters, such as non-spheroidal shape and ranges of aspect ratio. After sieving, visual inspecting can reasonably provide an estimate of per weight contributions. In most circumstances, the actual distributions at not near the limits of 50 percent by weight, so evaluation is straightforward with a significantly larger fraction by weight of the particles having the parameters.

The particle sizes can be selected for use in particular applications. The processing can be used to size separate a desired particle size range, as described in the following section. In general, thinner sheets are used when smaller particles are desired and thicker sheets are used when larger particles are desired. The particle sizes are selected by sieving, and the pre-grinding dimensions, the grinding speed and grinding time can influence the properties of the ground particles. Specific sizes useful for treatment of hypervascular arteries associated with inflamed joints are described in the next paragraph. The embolic particles can be distributed in different size ranges for different applications. For example, Embosphere® embolic particles (Merit Medical) are sold in seven different particle size ranges (50-100 microns, 40-120 microns, 100-300 microns, 300-500 microns, 500-700 microns, 700-900 microns and 900-1200 microns), and Embozene® microparticles are sold in 10 particle size ranges based on the average particle sizes (40 microns, 75 microns, 100 microns, 250 microns, 400 microns, 500 microns, 700 microns, 900 microns, 1100 microns, 1300 microns). In contrast, plate-shaped particles may be characterized by both the plate thickness and the sieve dimension. Sieving, as described below, can be used to select reasonable particle distributions for a particular use, and selected size ranges can be separately distributed.

For use in embolization of target small blood vessels associated with parent arteries, such as vessels associated with inflamed joints, particles can be grouped as passing through a 63 micron sieve, or bounded by 63 micron sieve on the lower end and a 250 micron sieve on the high end. Generally, smaller particles are separated using a 63 micron sieve. Sieves generally are sold in standard sizes, and a 63 micron sieve is a standard #230 US mesh size. Larger granules can be separated using a 250 micron sieve, which is a standard #60 US mesh size. Sieving for particle separation though is determined by spacing between holes in the mesh. Larger particles can be further ground to a size that fits through the 250 micron sieve. For testing purposes in the examples, samples passing through the 63 micron sieve are tested along with particles the pass the 250 micron sieve but are retained by the 63 micron sieve, 63-250 micron particles. Additional sieving can be performed to get smaller particle size distributions if desired, although the weight percent of particles produced in tighter ranges correspondingly decreases. As noted above, different particle size ranges can be prepared for different application of the embolic particles if desired.

Desirable particles can be formed from thin dense sheets of gelatin, generally salted gelatin, that have plate shapes following grinding. It is believed that most of the particles formed using this approach have a dimension corresponding to the thickness of the dried gelatin sheet prior to grinding. Larger particles have a plate shape with the original thickness corresponding to the sheet thickness and an expanse across the plate corresponding determined by the grinding. Smaller particles may have a more columnal shape with the thickness forming flat tops to the column and the walls resulting from the grinding process. While some particles may have fracturing along the thickness of the original sheet of dried gelatin, it is believed that such particles are a small percentage under grinding conditions generally used to maintain a moderate average particle size. Due to the enormous numbers of particles based on their small size, any manual review of the particle sizes can only examine a relatively small sampling. Rough particle sizes can be determined in a suspension for the hydrated particles based on dynamic light scattering experiments.

Hydration though swells the particles, generally to several times their dry size. The amount of swelling may depend on the initial density, the degree of crosslinking and the hydration time. Data suggests that the particles are well hydrated and are substantially fully hydrated in less than 30 minutes. Thus, particle size measurements made at after about 25 minutes can be assumed to be fully hydrated for particle size measurement purposes. The other features can be independently measured so that particle sizes can be considered in the context of characterizing the particles. Hydrogel swelling, and particle swelling for hydrogel particles, generally involves equal swelling of the material in all dimensions. Nevertheless, the swelling is not expected to significantly alter the particle shape.

Dynamic light scattering (DLS) experiments can be performed using commercial equipment and corresponding software analysis. This analysis generally is based on spherical particles, so dynamic light scattering generally does not provide information on particle shape, although it does provide information on distribution of particle sizes. Presumably, the particles are randomly oriented in a dilute suspension used for the DLS experiments, so an average projection of the particle sizes should roughly correspond with the measured value.

As presented in the Examples below, data is presented on the D₁₀, D₅₀ and D₉₀ from DLS experiments using exemplified particles after sieving. D₁₀ is the particle size in which 10 volume percent of the particles are below that size, D₅₀ is the particle size in which 50 volume percent of the particles are below that particle size, and D₉₀ is the particle size in which 90 volume percent of the particles have smaller particle sizes. DLS directly measures particle sizes according to volume fractions.

Due to sieving, the distributions are steep near the sieve values. In some embodiments, the sieving is performed using sieve sizes of 63 microns and 250 microns. This sieving using the particle processing described herein can result in D₁₀ values from about 140 microns to about 275 microns and in further embodiments form about 150 microns to about 250 microns, in D₅₀ values from about 285 microns to about 450 microns and in further embodiments from about 300 microns to about 440 microns, and in D₉₀ values from about 540 microns to about 700 microns and in further embodiments form about 550 microns to about 690 microns. A person of ordinary skill in the art will recognize that additional ranges within these explicit ranges above are contemplate and are within the present disclosure,

The density of the particles can be evaluated using displacement in a liquid where the particles do not dissolve or dissolve sufficiently slowly to obtain a volume measurement. Then, the weight of the particles divided by the volume provides the density. As noted above and explained further below, the process conditions are selected to produce dense particles generally free of pores in a dry state. The particles are measured to have a density of about 1.27 g/mL. This value is slightly below the reported gelatin density of 1.3-1.4 g/mL, see GMIA-Gelatin Handbook, 2012, incorporated herein by reference. Due to the presence of the salt, the particles hydrate effectively and quickly, and the swelling of the particles results in a decrease of the particle density. There may be small amounts of trapped gas or some minor pore formation that decreases the density, but that is not clear.

With respect to composition, the gelatin is characterized by the molecular weight of the initial gelatin powder. The gelatin in the powder are covalently bonded amino acids that are generally maintained during processing, although they are thermally crosslinked in the processing to make the particles. The molecular weight of the gelatin peptides has been found to relate to the mechanical strength of the gelatin, which can be measured in a Bloom Test. The Bloom test is similar to a durometer test, where a probe is used with a certain weight to deflect the gel without penetrating the gel. The Bloom test is well established in the art. The molecular weight of the gelatin generally can be from about 20,000 g/mole to about 100,000 g/mole, and the Bloom numbers can extend from about 30 to about 325. In some embodiments, the gelatin used has a Bloom number from about 100 to about 325, and in further embodiments from about 150 to about 325, corresponding to somewhat larger molecular weights over these ranges. A person of ordinary skill in the art will recognize that additional ranges of molecular weights and Bloom numbers within these explicit ranges are contemplated and are within the present disclosure.

The gelatin should be appropriately free of endotoxins. While commercial gelatins can be further purified to reduce endotoxin levels, commercial gelatin powder can have desirably low endotoxin levels as sold. Generally, the endotoxin levels can be made as low as needed through additional purification with an associated increase in cost. Generally, the gelatins used for making embolic particles have appropriate endotoxin levels, and regulatory limits generally guide suitable values.

As noted above, salt is added to improve the particle properties. Generally, any soluble and biologically acceptable salt can be used, and these are generally metal halides. Chloride ions are prominent in medicinal salts, although low concentrations of bromide can be acceptable and small levels of fluoride may be acceptable. For simplicity and broad acceptance, sodium chloride can be used as the salt, although calcium chloride at relevant amounts would likely also be appropriate and some other metal cations may be acceptable in low concentrations. While other salts can be used to influence the gelatin properties, sodium chloride is commonly used for intravenous use and would be the appropriate salt for acceptable introduction into a patient. Any other salts that are sufficiently non-toxic can be used. The amount of salt can be from about 1 weight percent (wt %) to about 66 wt % of the dried particles, in further embodiments from about 1.5 wt % to about 50 wt % and in other embodiments from about 2 wt % to about 40 wt % relative to the total particle weight. Due to the higher density of sodium chloride (2.17 g/mL) relative to gelatin, the volume percent contribution of the salt is correspondingly less. A person of ordinary skill in the art will recognize that additional ranges of salt amounts within the explicit ranges above are contemplated and are within the present disclosure.

In vivo placement of gelatin particles that have uncrosslinked gelatin dissolve very quickly due to solubility of the gelatin at elevated temperature. Gelatin can be chemically crosslinked using a chemical crosslinking agent, such as dialdehydes. Chemical crosslinking agents can introduce toxic agents, especially if the crosslinking is not performed to form essentially a non-degradable highly crosslinked material. Thermal crosslinking can be used to avoid introduction of potentially toxic agents from chemical crosslinking while stabilizing the gelatin for desired degradation times. The length of time and temperature of thermal crosslinking influences the degradation times. While not wanting to be limited by theory, it is believed that thermal crosslinking can result in the crosslinking of suitable groups that are nearby in the structure. The thermal crosslinking procedure is described further below. The degree of crosslinking is evaluated herein based on the digestion time in an in vitro test. The in vitro digestion times correlate qualitatively with expected in vivo degradation times and recanalization rates.

The in vitro degradation time may be controlled using thermal conditioning, particle size and aspect ratio as the particles digest in pepsin or dissolve in PBS through surface erosion primarily. If one dimension is small in relationship to the other two dimensions, it will digest/dissolve faster than a particle with the same thermal conditioning/crosslinking with a different aspect ratio. In addition, making a thinner sheet, such as with a nominal 30 μm thickness, for grinding may produce more smaller particles, <63 μm, relative to a thicker sheet, with a nominal 100 μm thickness. A thicker sheet on the other hand would allow for a larger batch size compared to a thin sheet when preparing larger particles.

References to pepsin digestion times herein refer to the use of the following process. To evaluate digestion times, a solution is prepared comprising 0.5 mg/ml of pepsin in 10 mM HCl(aq). Pepsin is a proteinase that degrades proteins such as gelatin and collagen. A 20 mg quantity of gelatin particles, generally following desired sieving, are placed into 20 ml of the pepsin solution. The digestion time was set as the time at which the particles were fully digested to the eye, i.e., visually. The digestion times were influenced by the crosslinking times and temperatures. The digestion times could be established from about 2 minutes to about 48 hours, in further embodiments from about 5 minutes to about 24 hours and in other embodiments from about 10 minutes to about 12 hours. In vivo recanalization times are observed to be significantly greater than the in vitro measurement and may depend on the particular vessels involved. A pepsin digestion time of an hour can correspond with an in vivo recanalization time on the order of weeks or a couple of months. It can be useful to contemplate embolic products with selected different ranges of digestion times directed to particular medical applications. A person of ordinary skill in the art will recognize that additional ranges of digestion times within the explicit ranges above are contemplated and are within the present disclosure.

The digestion rates measured in the above in vitro test generally would correlate with in vivo absorption rates. Similarly, observed recanalization rates are correlated with absorption rates. Generally, recanalization may be observable in x-ray imaging, and absorption rates may be inferred from these observations. The correlation between in vitro digestion times and in vivo absorption times may depend on the particular blood vessel(s) involved. In some embodiments, desirable ranges of recanalization rates can range from about 10 minutes to about 60 days, in further embodiments from about 20 minutes to 45 days, in additional embodiments from about 30 minutes to about 30 days, in further embodiments from about 45 minutes to about 20 days and in other embodiments from about 1 hour to about 10 days, as well as any range exchanging the upper and lower bounds of these specific ranges, such as from about 10 minutes to about 10 days. A person of ordinary skill in the art will recognize that additional ranges of recanalization rates within the explicit ranges above are contemplated and are within the present disclosure.

Test provide in vitro evidence that ellipsoid shaped particles can pack with a low leakage configuration. Spherical particles have a natural limit to their packing density. For particles of equal size there are closest packing configurations that can be characterized. Due to the shape of spherical particles, gaps between the particles generally make it difficult to prevent leaks through a vessel blocked with spherical particle, so occlusion generally is incomplete due to leakage. On the other hand, very irregularly shaped particles can have appendages and other irregularities that make a tight packing of the particles difficult or impossible. The ellipsoid shaped particles described herein can provide orderly and complete packing with low or eliminated leakage. In addition, the shape of the particles tends to orient the particles in a flow. So after the ellipsoid shaped particles are delivered, they tend to orient in the flow with a reduced tendency to disperse in the flow. So the ellipsoid shaped particles tend to follow the flow and orient appropriately to form a well packed occlusion with little or no leakage. Thus, occlusions with eliminated leakage are a further characteristic of the use of particles described herein using the processing described herein.

### Gelatin Particle Formation

The gelatin particles are formed through the grinding of thin sheets of dried salted gelatin, and the salt is added to help to control the particle properties. As noted above, there are two ways to form the salted gelatin sheets, one based on a dense gelatin film and a second based on foamed gelatin sheet. The use of the dense gelatin layers can result in a desirable plate shape of the ground particles if the processing parameters are chosen to produce plate shaped particles. Either processing approach results in effective embolic particles. The processing can be considered to have a few stages. Once salted gelatin layers are formed, the grinding and sieving processes are similar for the two approaches.

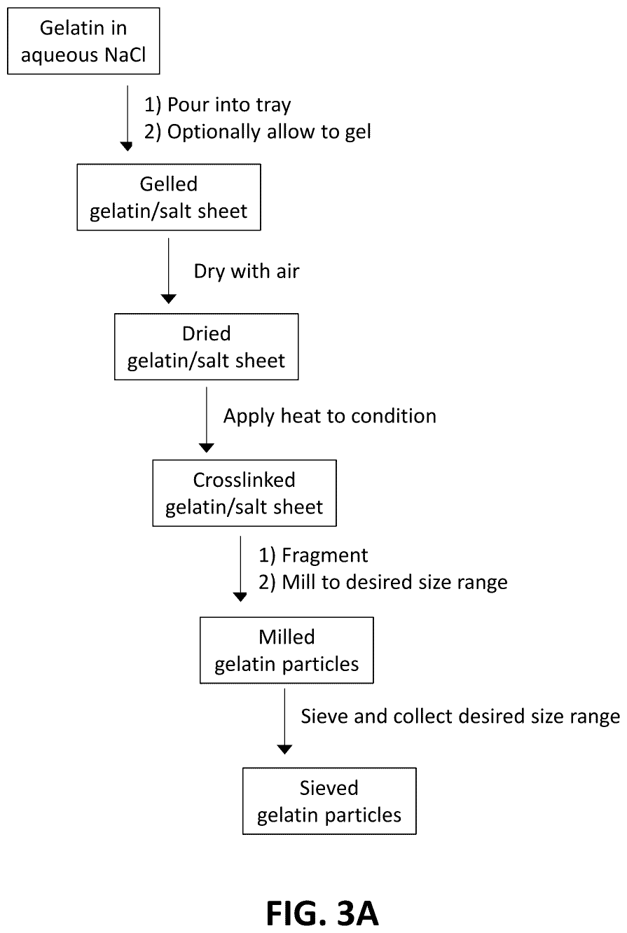

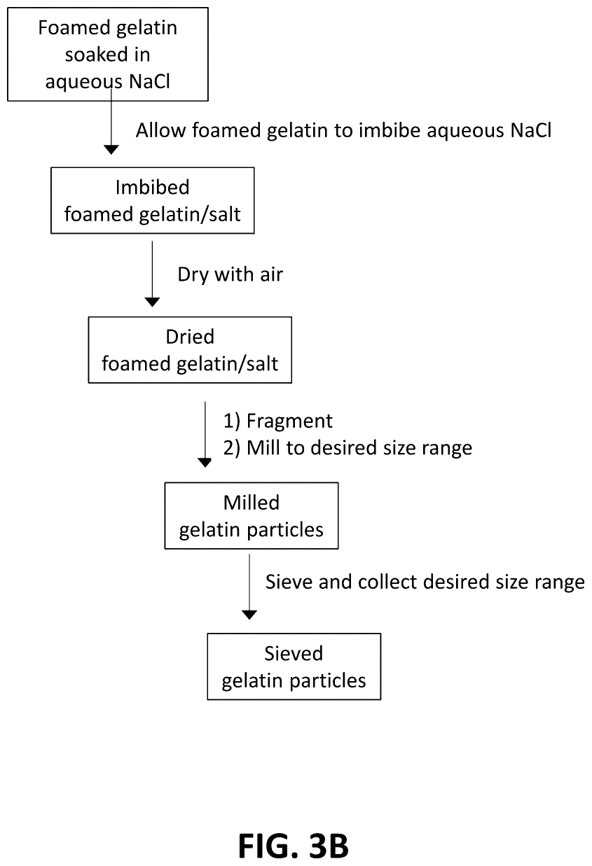

FIG. 3A depicts a general flow procedure for forming embolic particles from salted gelatin according to an embodiment of the present invention. Powdered gelatin or other suitable soluble gelatin particles, is added to an aqueous salt solution comprising NaCl to form a gelatin/salt solution. The aqueous salt solution can comprise NaCl at concentrations ranging from about 0.05M (about 0.3% w/w) to about 1M (about 5.8% w/w) and can be warmed to a suitable temperature in order to facilitate dissolution of the gelatin. Dissolution is complete once schlieren is no longer visible upon stirring and/or shaking. The concentration of the gelatin can be at least about 0.5% (w/w).

An amount of the gelatin/salt solution is poured into a tray and optionally allowed to gel upon cooling. The amount of gelatin/salt solution added to the tray depends upon the dimensions of the tray. For example, for a tray having an area of about 600 cm², the amount of gelatin/salt solution can range from about 50 g to about 500 g, although for commercial production, larger trays or the like can be contemplated. The thickness of an as-deposited gelatin layer following drying depends on the concentration. For example, a 300 gram deposit of a 1 wt % solution results in a dry thickness of 33 microns, while a 90 gram deposit of a 10 wt % solution results in a 100 micron dry average thickness. The resulting gelled gelatin/salt sheet is then dried with flow of air or other gas, such as nitrogen, at temperatures below the melting point of the gelatin gel which is typically about 34° C. for a high Bloom gelatin. In alternative embodiments, the gel remains in a liquid state (melted) during drying. The resulting dried gelatin/salt sheet is then conditioned such that the gelatin becomes crosslinked. As described above, conditioning can comprise thermal conditioning by applying heat at temperatures ranging from about 100° C. to about 180° C.

The resulting crosslinked gelatin/salt sheet is then removed from the tray and fragmented or broken up into large pieces. The sheet can be fragmented manually. The large pieces are then ground or milled, then sieved to obtain a desired size fraction which is weighed. Milling can be carried out using any suitable type of mill such as a knife mill, and parameters such as rpm and time are not particularly limited as long as the milled gelatin particles can be sieved to produce an acceptable yield within the desired size range. Milling is described above and in the Examples. Useful particles size ranges for the desired size fraction are described above and in the Examples.

Due to the relative thinness of the sheet, small deviations from flatness in the trays can result in thickness variation across the sheet. The quantity of gelatin can be targeted for the desired average thickness. The thickness variation does not interfere with particle production or with particle performance for occluding vessels.

For embodiments of gelatin platelets formed by grinding dry sheets of gelatin or fragments of the sheets, the average thickness of the platelets roughly corresponds to the average thickness of the sheet that is ground to make the particles. The grinding process generally, fragments the sheets to form platelets with surfaces corresponding to the top and bottom of the sheets. So the grinding can fragment particles with an x-y planar surface that are larger than the thickness, which can be referred to as a z-direction. Due to a roughly planar surface with dimensions significantly greater than the thickness, the shape is referred to as platelets, and the term is explained further above. Of course, grinding is not a precise process, so some fraction of the weight of the particles have alternative particle shapes with some small particles produced as by products. Generally, at least half of the particles by weight have a platelet shape, with the particle size distribution depending on the grinding process and target weight average particle size.

FIG. 3B depicts a general flow procedure for forming embolic particles from salted foamed gelatin according to another embodiment of the present invention. The gelatin foam or sponge is generally lightly crosslinked prior to commencing processing to stabilize the foam structure. The gelatin foam is contacted with an aqueous salt solution comprising NaCl or other suitable salt such that the gelatin foam imbibes the solution. The foamed gelatin can be a foamed gelatin sheet or sponge. The aqueous salt solution can comprise NaCl at concentrations ranging from about 0.05M (about 0.3% w/w) to about 1M (about 5.8% w/w).

The imbibed foamed gelatin/salt sheet or pieces are dried with blowing air or other gas at a suitable temperature. The dried foamed gelatin/salt material is fragmented or broken up into pieces that are then milled and sieved to obtain a desired size fraction as described above. Dry particles formed with this processing generally have platelet shapes, which are surprisingly similar to the shapes of the particles formed from the dry dense salted gelatin. As demonstrated below, particles formed from grinding the foamed gelatin without salting have a very different shape that does not look like platelets.

With respect to the formation of dense salted gelatin sheet, a gelatin powder or granules are used as a starting material. The gelatin can be obtained from a suitable gelatin supplier. Medical grade gelatin is available commercially from, for example, Gelita Medical and Rousselot. The obtained commercial gelatin can have a particular molecular weight average and endotoxin level. Further purification can be performed to obtain a lower endotoxin level. Gelatin is soluble in warm water.

The gelatin is first dissolved in warmed saline to form a solution that is transferred to appropriate trays or the like to form a coating of gelatin that forms a gel upon cooling. The gel is then dried to form a dried film of salted gelatin. To form particles, the dried film is ground and sieved to obtain the particles. Features of the dry gelatin sheet formation are selected to provide desirable properties of the ultimate particles. The formation of the dried sheets influences the successful grinding and sieving process to produce the appropriate particles having properties useful for injection into patients.

Specifically, the gelatin can be dissolved in water at a temperature from about 35° C. to about 70° C. at a concentration from about 0.5 wt % to about 15 wt %. While the salt can be added later in the process, it can be desirable to dissolve the salt in the water prior to adding the gelatin. Thus, the warm water into which the gelatin is dissolved should include the desired amount of salt targeted for the final salted gelatin particles. The solutions should be well mixed, but forming the solutions is generally unremarkable.

The solution of dissolved gelatin and salt is poured into trays at a desired thickness. The thickness of the poured solution influences the thickness of plate shaped particles, which is a function of the wet thickness and the solid content. The thickness is generally selected to yield an average dry thickness of no more than about 2000 microns, in some embodiments from about 20 microns to about 2000 microns, in further embodiments from about 25 microns to about 1500 microns, in some embodiments from about 25 microns to about 1000 microns, in additional embodiments from about 25 microns to about 500 microns, and in other embodiments from about 30 microns to about 200 microns. The upper and lower limits of these ranges can be exchanged, such as to form a range from about 30 microns to about 2000 microns. The trays generally have some deviation from flatness that results in corresponding thickness variation of the gelatin sheet. In some embodiments, the thickness variation resulting from deviations from flatness of the trays or other causes can be no more than about ±90 microns, in further embodiments no more than about ±50 microns and in other embodiment no more than about ±25 microns. More precisely constructed plates can reduce this variation. These deviations from flatness are over the whole expanse of the tray, so that thickness variation of individual platelets generally are small, but uniformity of platelet thickness between platelets generally reflects this variation. The sheet thicknesses can be selected to achieve a particle distribution from grinding that achieves an appropriate yield of particles in a desired size range. Regardless, particles can be size separated to contribute to potentially alternative product sizes of different particle size ranges are produced. The nature of the trays is not important as long as they are inert to the salted gelatin solution, provide for forming and removing the desired sheet of dry gelatin, and tolerate low heating. The trays should have a low surface energy so that the gelatin sheets can be removed from the trays after drying. The thermal crosslinking may or may not be performed in the same tray depending on the heat tolerance of the drying trays. In the examples below, the dried gelatin sheets are removed and placed into a metal tray for thermal crosslinking. The gelatin in the tray forms a gel upon cooling from the temperature used to dissolve the gelatin. While not wanting to be limited by theory, it is thought that the gelling process can orient the polymer chains in the gelled material which is then dried and influences the particle properties after grinding. In particular, the digestion times for gelatin dried from a gelled state have significantly longer digestion times relative to gelatin dried from a melted state. A person of ordinary skill in the art will recognize that additional ranges of dry thicknesses within the explicit ranges above are contemplated and are within the present disclosure.

The gelatin in the trays is dried. The density of the dry gelatin is about 1.5 g/mL. Due to the large surface area of the gelatin in the trays, the gelatin dries relatively quickly and effectively. Generally, room temperature or slightly warmed air can be blown across the plates. Room air, dried air, nitrogen or the like can be used if desired for drying. The gelatin in the trays can be susceptible to melting into a liquid from the gelled state if warmed. Generally, gelatin melts around 34° C., but highly salted gelatin is observed to melt at a temperature roughly one to a few degrees lower. To avoid melting, the drying air/gas can be slightly heated, generally to less than 33° C. or comparably slightly lower than the melting temperature. While not wanting to be limited by theory, in the gelled state, the gelatin may form hydrogen bonds aligning potentially cross-linkable moieties, which can lead to desirable particle properties if a higher degree of thermal crosslinking and corresponding longer digestion times is desired. But drying the gelatin from a melted state can nevertheless result in suitable gelatin particles for use as embolic particles with a relatively more rapid digestion time. So if drying a melted gelatin is desired, the tray or gas over the tray can be heated sufficiently to melt the gelatin. In any case, it is desirable for the drying process to be relatively slow to facilitate forming a dense dry gelatin and to avoid thermal crosslinking until the drying process is completed. The drying process is continued until the moisture is effectively removed keeping in mind that the polymer may retain a small amount of bound water.

With respect to processing of foamed gelatin sheet, generally an open cell foam, the material is cut to a desired size including the thickness. The salting is performed by imbibing a saline solution into the porous sheets, which can involve some compression and re-expansion to facilitate incorporation of the saline. A gelatin foam starting material can be lightly crosslinked to facilitate stability of the foam. This light crosslinking also tends to keep the gelatin foam from melting. The concentration of saline in the salting solution should be selected to provide a desired amount of salt for the ultimate particles. The ranges of degree of salting is described above. In some embodiments, the volume of salting solution can be selected to just cover the foamed gelatin once the solution has completed wetting of the foam to provide a more uniform salt distribution, although uniformity is not particularly significant. After depositing the salt solution and allowing the salt solution to fully wet the thickness of the foam, the gelatin sheet is dried. The drying can again be performed by blowing gas, such as air, gently across the gelatin. No heat or only a small amount of heat can be blown for the drying. Once the sheet is dried, a salted gelatin foam remains for further processing into particles.

After drying, the layers of gelatin, either dense or foamed, can be subjected to thermal crosslinking. Gelatin foam generally is lightly crosslinked prior to salting, and additional thermal crosslinking after salting may or may not be used. The degree of thermal crosslinking significantly influences the digestion time in in vitro testing and is correlated to the in vivo absorption times and the recanalization times. In general, thermal crosslinking can be performed at temperatures from about 100° C. to about 200° C., and in further embodiments from about 120° C. to about 190° C. for time from about 15 minutes to about 48 hours, in further embodiments from about 25 minutes to about 36 hours, in some embodiments from about 30 minutes to about 32 hours and in additional embodiments from about 35 minutes to about 24 hours. A person of ordinary skill in the art can adjust the time and temperature to achieve desired digestion properties based on the teachings herein. A person or ordinary skill in the art will recognize that additional ranges of thermal crosslinking parameters within the explicit ranges above are contemplated and are within the present disclosure.

Following thermal crosslinking, the gelatin layers are ready for particle formation. Various suitable commercial mills are available to perform the grinding. For example, a knife mill or a rotor mill. The salted gelatin sheets can be placed into the mill. Mill instructions can be followed to perform the desired degree of grinding. While grinding parameters are adjusted for a specific mill, specific gelatin sheet properties and specific target particles, generally mill rates can be form about 1000 rpm to about 30,000 rpm, and grinding time can range from about 2 seconds to about 1 hours and in further embodiments from about 15 seconds to about 40 minutes. Similarly, commercial sieving devices can be used to efficiently and reproducibly perform the sieving. Commercial sieving shakers provide standard sieve sizes and mechanical shaking for processing of materials using the sieves. The instructions for the commercial sieves and be followed to perform the sieving function in terms of capacity and timing for the sieving process. A person of ordinary skill in the art will recognize that additional ranges of milling rates within the explicit ranges above are contemplated and are within the present disclosure.

At an appropriate time of the production process, the gelatin particles can be sterilized using gamma irradiation, although potentially alternative sterilization approaches could be used. For medical use the particles should be prepared under current good manufacturing practices as specified by the FDA or other appropriate regulatory agency, and then sterilized, for example, in the packaging. Gamma irradiation is very penetrating and is an accepted process for medical devices. As shown in the Examples, gamma irradiation does not damage the gelatin particles to render them unsuitable for use. Small modification of the digestion times may occur as a result of gamma irradiation, but the changes are small and can be adjusted for accordingly. Thus, the gelatin particles described herein are suitable for gamma sterilization.

### Use of Gelatin Particles

Embolic microparticles can be used for various indications. The discussion below provides further details regarding the specific use for embolizing vessels associated with inflammation of blood vessels associated with joint pain. For use as embolic particles, the particles are generally mixed into a dispersion or suspension. To facilitate visualization, embolic particles are generally suspended in a solution comprising radiopaque contrast dye that block x-ray radiation, although other suitable liquids for vascular infusion could be used to suspend the particles for delivery. IV saline, water for injection or similar sterile liquids can be used to suspend or adjust the concentration of the particle dispersions/suspensions. The suspension is generally then injected and delivered into a blood vessel adjacent to a target location.

Appropriately biocompatible contrast dyes are commercially available. For use in the vascular system, contrast agents generally comprise iodine. Iodine containing compositions for use as contrast agents include, for example, iohexol, iodixanol, ioversol, which are sold under various brand names. The contrast agents can be provided with different levels of iodination. For use, it is useful to form a mixture of contrast agent with roughly 50 volume percent (±10 vol %) sterile saline, although other proportions of contrast and saline can be used as desired, such as from about 25 vol % saline to about 75 vol % saline. A person of ordinary skill in the art will recognize that additional ranges of saline volume percent concentrations within the explicit ranges above contemplated and are within the present disclosure.

The suspension is formulated using a desired amount of embolic particles for the particular application and mixed with an appropriate amount of liquid to deliver the appropriate amount of volume into the vessel. The injected volume of liquid is generally from about 0.5 ml to about 10 ml. The quantity of embolic particles is generally from about 5 mg to about 500 mg, in further embodiments from about 10 mg to about 400 mg, and in other embodiments from about 12 mg to about 250 mg. A person of ordinary skill in the art will recognize that additional ranges of volumes and embolic particle weight within the explicit ranges above are contemplated and are in the present disclosure.

For distribution, the particles can be shipped dry to extend the shelf life. For other embodiments, the embolic particles can be shipped in saline, contrast dye or a mixture thereof. If shipped dry, the embolic particles can be mixed with the liquid prior to use. The particles can be shipped in a vial or in a syringe. If shipped in a particular liquid, additional liquid, such as contrast dye can be combined to form the desired injection composition. Once combined with the liquid, the suspension should be well mixed and placed in a syringe if not mixed in a syringe, although another delivery container can be used as appropriate. Generally, an appropriately sized syringe is used for the desired volume of fluid for delivery.

A catheter is inserted into the patient using conventional hemostatic placement generally through an appropriate introducer sheath or the like. A catheter is inserted through the vasculature through the introducer and placed with the distal end of the catheter at the selected position for embolic particle delivery. Once in position, the syringe can be used to deliver the embolic particles into the vasculature. Positioning the catheter, delivery of the particles and monitoring of the efficacy of the particles can be monitored in real time using x-ray imaging in conjunction with a contrast agent.

As noted above, the average particle size and degradation times can be selected and adjusted for the particular indication addressed by the delivery of the particles. The particle size should be selected based on the size of the vessel to be occluded and whether proximal or distal occlusion is desired. Existing embolic particles are approved for hypervascular tumors, such as hepatoma and symptomatic uterine fibroids, prostatic arteries for symptomatic Benign Prostatic Hyperplasia (BPH), arteriovenous malformations, and blood vessels to occlude blood flow to control bleeding/hemorrhaging in the peripheral vasculature.

As noted above, embolic particles have been used for hypervascular arteries associated with inflammation occurring with joint pain. Such treatments are discussed above in the context of the Okura references and the '402 application. The embolic gelatin particles described herein provide a significant improvement over the exemplified starch particles of the '402 application and the antibiotic particles of the Okura references for temporary occlusion. With respect to longer lasting particles, the platelet/ellipsoidal shapes and the mechanical properties of the salted gelatin particles described herein provide improved packing in the vessels relative to the synthetic spherical particles commercially available. The gelatin particles form hydrogels upon hydration, which are relatively soft and conformable, especially if not too highly crosslinked. As noted above, the degradation times can be selected over a broad range.

A hypervascularized tissue is characterized by a network of blood vessels that starts as a branch from an artery that is essentially normal in appearance. The branch gives rise to further branches and/or fine blood vessels. The fine blood vessels are visualized as a “blush” on an angiogram using radiopaque compounds for visualization in a manner customary in these arts. It is believed that the elimination of the fine vessels can be adequate for treatment of pain associated with hypervascularization and that it is not necessary to place embolic particles to block flow in the larger of the branches. Therefore, treatments can be directed to avoiding embolization of the relatively large branches while embolizing the fine branches. Undesirable side effects that result from targeting the relatively larger branches can then be avoided. As a part of this proposed approach, fast-degrading materials are used to embolize fine vasculature so that there is no, or little, recanalization, i.e., the effects of embolization are long lasting for the fine vessels. The same fast-degrading materials may temporarily block flow in larger vessels without compromising the efficacy of the treatment and also without causing harmful side effects that result from treatments that target the relatively larger vessels for longer term blockage. Further, materials may be used that are absorbable to leave only biocompatible residues, which is a term used herein that means residues of an embolic material that are soluble components that can be locally cleared by dissolution into blood and eventually systemically cleared over time by excretory mechanisms.

To reduce inadvertent adverse events, embolic particles should be properly selected to degrade in to allow recanalization of larger vessel to minimize the negative effect of any off target embolization. Methods that embolize the relatively larger vessels with permanent embolic particles in the context of treating hypervascularity in response to chronic inflammation have resulted in observed harmful side effects such as skin necrosis/color change, peripheral paresthesia/numbness, and one or more of muscle weakness, dullness, and pain. These unwanted and harmful side effects may be reduced or eliminated with proper size selection and use of the embolic particles described herein.

### EXAMPLES

### General Methods and Materials

### Preparation of Gelatin/Salt Sheets

Gelatin/NaCl sheets were prepared as follows:
1. Dissolve NaCl in high purity water at NaCl concentrations ranging from about 0.5% (w/w) to about 10% (w/w).
2. Dissolve gelatin in aqueous NaCl at gelatin concentration 1-10% (w/w) at elevated temperature, 40-70° C.
3. Pour an amount of gelatin/NaCl solution in tray and optionally allow gelatin to form a gel.
4. Dry at around 30° C. in an environment with convection. If the aqueous gelatin solution was allowed to form a gel, care is taken not to melt the gel which has a melting temperature of about 34° C.
5. Condition (crosslink) the gelatin at a temperature from 100-170° C. for a selected period of time.

In the creation of the gelatin/salt sheets the concentration and the amount of gelatin/NaCl solution added to the tray controlled the thickness of the dried sheet. The thickness of the sheet may, to a large part, control one dimension of the particle, i.e., a thin sheet may produce overall smaller particles. For example, a 1% (w/w) gelatin solution can be used to produce a gelatin/salt sheet with an approximate thickness of about 50 μm and a 10% (w/w) gelatin solution can be used to produce a gelatin/salt sheet with an approximate thickness of about 150 μm. To achieve a desired dry gelatin thickness, the concentration and/or the wet thickness can be adjusted accordingly.

The gelling of the gelatin and low temperature drying improved the film formation by producing more consistent film thickness. While not wanting to be limited by theory, the gelling process seems to improve the crosslinking process as the gelling may cause the individual gelatin molecular moieties to arrange in a way as to increase the crosslink density from thermal crosslinking as compared a gelatin/salt material dried in the liquid state where the conformation may be more random when exposed to heat for crosslinking. Particles produced with melted samples that are dried had shorter pepsin degradation times, which seems to suggest a lower degree of crosslinking.

The gelatin/salt particles generally had improved wetting characteristics as compared to gelatin particles prepared without salt as the gelatin/salt particles were found to wet and disperse faster in the various solutions compared to gelatin particles prepared without salt. The addition of NaCl may counter the somewhat hydrophobic nature of gelatin which consist of long hydrophobic segments with dispersed short hydrophilic segments in its structure. While gelatin generally is water soluble, the hydrophobic domains moderate the hydration process and addition of a salt improves solubility as compared to deionized-water.

### Preparation of Gelatin/Salt Particles

Gelatin/salt particles were prepared by grinding the dried gelatin/salt sheets. Grinding was carried out using a commercial knife mill.

The addition of a NaCl to gelatin provided improved processing of the gelatin/salt samples compared to gelatin without salt. The improved processing included easier grinding into particles and improved sieving characteristics. The grinding was improved due to increased brittleness of the gelatin/salt samples compared to gelatin alone. In the gelatin/salt samples, the static charge may be reduced compared to gelatin alone, facilitating fast and complete sieving of the particles. The reduced problems of static charge may be due several factors such as increased density, less gelatin as salt is added, less buildup of static charge and/or improved dissipation of static charge. The processing characteristics were improved with increasing content of NaCl in the samples.

### Embolization Evaluation

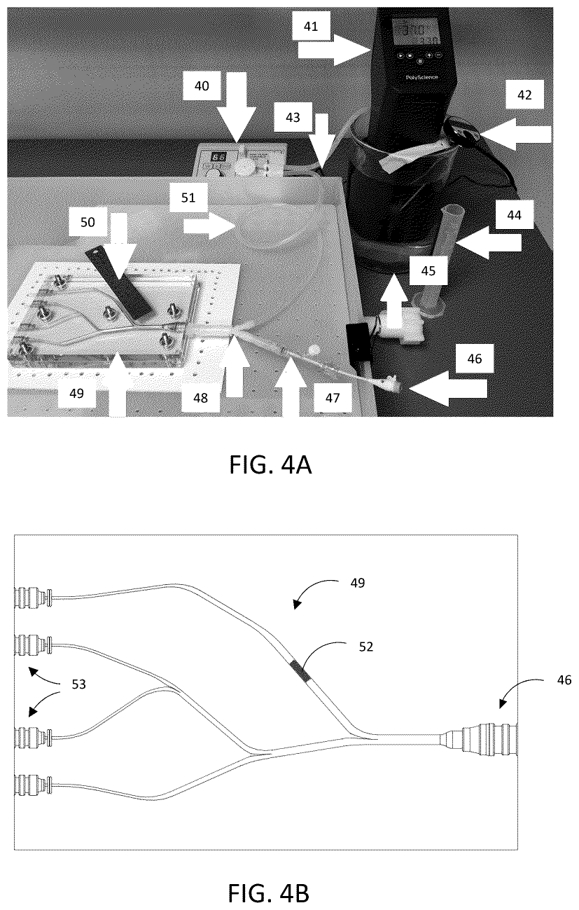

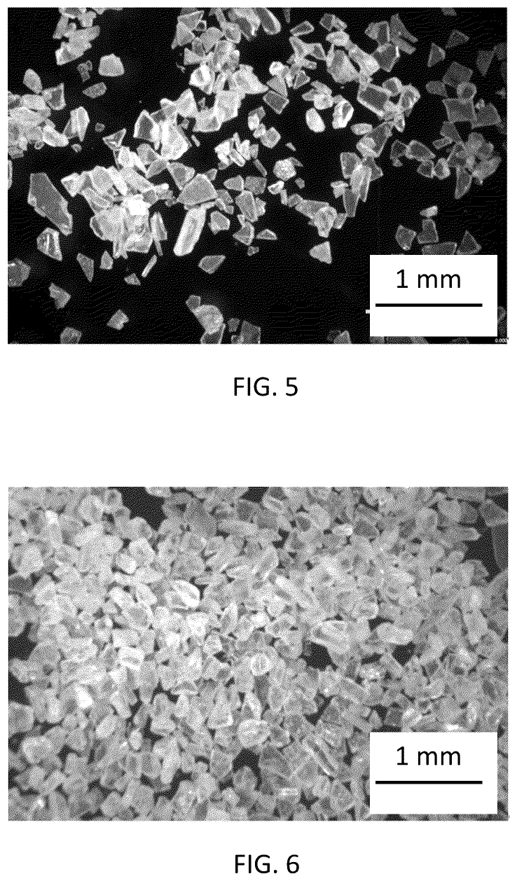

The gelatin and gelatin/salt particles were evaluated using a flow model with water at body temperature designed to mimic blood vessels with a parent artery of 4 mm gradually branching down to a 500 μm vessel size. The set up for the flow model is shown in FIG. 4A. Components 40-52 are described in Table 1.

TABLE 1

| Component | Description |
|---|---|
| 40 | Variable Flow Mini Pump; with ¼ in tubing set - |
| | Natural Colored O Ring Fittings Or |
| | variable speed peristaltic pump with ¼ in tubing |
| | set or equivalent |
| 41 | Water Bath Heater; (or equivalent), Set to 37° C. |
| 42 | Thermometer; (or equivalent), To confirm |
| | temperature of water reservoir |
| 43 | Intake Tubing; 0.188 in ID × 0.312 in OD × 20 in |
| 44 | 50 ml Graduated Cylinder; For flow rate |
| | measurement (or scale and beaker - not pictured) |
| 45 | Container; Water reservoir for flow loop. 2-5 liters |
| 46 | Introducer; |
| 47 | Hemostasis Valve; |
| 48 | Y Connector; Barb-barb, ⅜ in × ¼ in × ¼ in (or |
| | equivalent) |
| 49 | Vascular Model; 0.5-4 mm |
| 50 | Ruler; 6 in Stainless Steel, |
| 51 | Outlet Tubing; 0.188 in ID × 0.312 in OD × 30 in |
| 52 | 20 μm frits |
| 53 | Connecters such as luer connectors |

A schematic diagram of the vascular model portion of the model is shown in FIG. 4B. The diagram shows 5 French Introducer 46 for introducing an embolic material into Vascular Model 49 comprising tubing with diameters of from 0.5 mm to 4 mm in diameter. (For clarity, Hemostasis Valve 47 and Y Connector 48 are not included in the diagram.)

Approximately 100 g of ground particles were suspended in a solution mixture of saline and contrast agent solution. Conventional sterile saline for intravenous use is 0.9 wt % sodium chloride in water and in a medical context is routinely referred to just as saline. The contrast agent solution was Omnipaque™ 300 from GE HealthCare which is formulated to include iohexol at 647 mg/mL with organically bound iodine at 300 mg/mL. The solution mixture was 50:50 by weight of saline to contrast agent solution unless otherwise noted.

### Gelatin Materials

Gelatins from various sources with Bloom values corresponding to relatively high average molecular weights included the followed and are referred to as G-A to G-C:

| | |
|---|---|
| G-A | Supplier 1, Less Purified Sample |
| G-B | Supplier 1, More Purified Sample |
| G-C | Supplier 2 |

### Example 1—Embolization Evaluation

This example describes four specific embodiments. The last sample was formed without salt as a counter example.

To form a first sample, approximately 10 g of gelatin G-C was dissolved in 990 g of 0.1M aqueous NaCl at 60° C. to give a 1% (w/w) solution of the gelatin. Approximately 300 g of the solution was poured into a polypropylene tray (600 cm²) and allowed to gel. The polypropylene tray used in all examples measured 600 cm² which includes the lip around the perimeter of the tray). The gelled sample was allowed to dry at 31° C. with air flow to form a dried gelatin sheet with an average thickness of about 50 μm. The dried gelatin sheet was then subject to thermal conditioning in an oven at 140° C. for 3 hours. The dried gelatin/salt sheet was ground and sieved to a size range of 63-250 μm. Approximately 100 mg of the gelatin/salt particles were added to a syringe and suspended in 5 mL of the saline/contrast agent solution. Embolization was evaluated in the flow model described above and found to reach stasis.

To form a second sample, approximately 100 g of gelatin G-A was dissolved in 900 g of 0.41M aqueous NaCl at 60° C. to give a 10% (w/w) solution of the gelatin. Approximately 90 g of the solution was poured into a polypropylene tray (600 cm²) and allowed to gel. The gelled sample was allowed to dry at 31° C. with air flow to form a dried gelatin sheet with an average thickness of about 100 microns. The dried gelatin sheet was subject to thermal conditioning at 160° C. for 8 hours in an oven. The dried gelatin/salt sheet was ground and sieved to a size range of 63-250 μm. Approximately 100 mg of the gelatin/salt particles were added to a syringe and suspended in 5 mL of the saline/contrast agent solution. The embolic particles were evaluated and found to embolize the flow model.

To form a third sample, approximately 100 g of gelatin G-B was dissolved in 900 g of 0.41M aqueous NaCl at 60° C. to give a 10% (w/w) solution of the gelatin. Approximately 90 g of the solution was poured into a polypropylene tray (600 cm²) and allowed to gel. The gelled sample was allowed to dry at 31° C. with air flow to form a dried gelatin sheet with an average thickness of about 100 μm. The dried gelatin sheet was subject to thermal conditioning at 160° C. for 3 hours in an oven. The dried gelatin/salt sheet was ground and sieved to a size range of less than 63 μm. Approximately 100 mg of the gelatin/salt particles were added to a syringe and suspended in 5 mL of the saline/contrast agent solution. The embolic particles were evaluated and found to embolize the flow model. The performance observed in the flow model was comparable to the performance observed with the larger particles in Samples 1 and 2.

To form a comparative example, approximately 100 g of gelatin G-A was dissolved in 900 g of deionized water at 60° C. Approximately 90 g of the solution was poured into a polypropylene tray (600 cm²) and allowed to gel. The gelled sample was allowed to dry at 31° C. with air flow to form a dried gelatin sheet with an average thickness of about 150 microns. The dried gelatin sheet was subject to thermally conditioned at 160° C. for 8 hours in an oven. The formed gelatin sheet was ground and sieved to a size range of 63-250 μm. This sample was not tested in the flow model, but it is assumed that it would embolize the vessels in the model since embolization is still faster than absorption time.

### Example 2—Digestion Times for 63-250 μm Particles

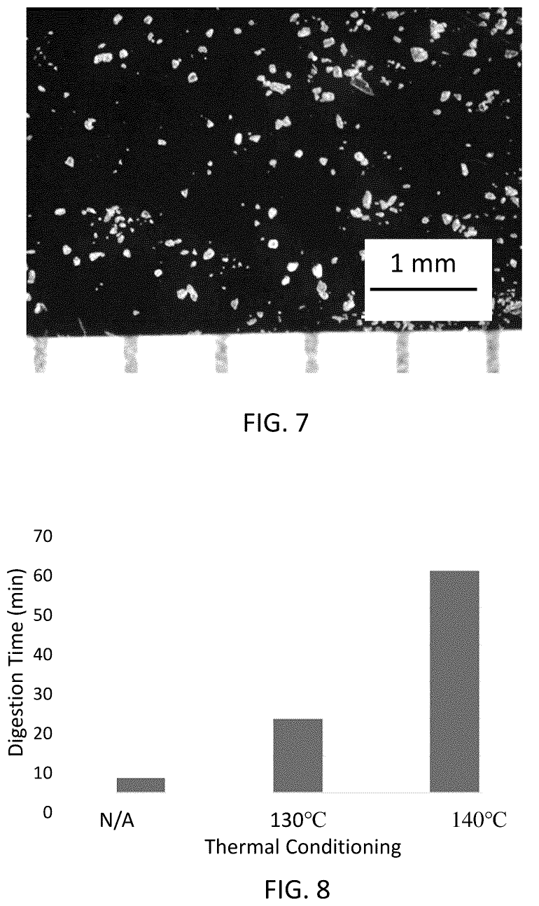

Gelatin/salt sheets having a thickness of about 50 μm were prepared with gelatins G-A and G-C. For each sheet to be formed, a 1% (w/w) solution was prepared by dissolving 10 g of gelatin in 990 g of 0.1M aqueous NaCl at 60° C. Approximately 300 g of the gelatin solution was poured into a polypropylene tray (600 cm²). Some gelatin solutions were allowed to gel in the tray then dried at 31° C. with air flow. Other gelatin solutions were not allowed to gel (remained in liquid state) and were dried at 31° C. with air flow. The dried gelatin/salt sheets were thermally conditioned at temperatures ranging from 140° C. to 180° C. and for times ranging from 4 hours to 24 hours. The conditioned gelatin/salt sheets were broken up by hand then ground and sieved to a size range of 63-250 μm. An optical micrograph of example particles is shown in FIG. 5.

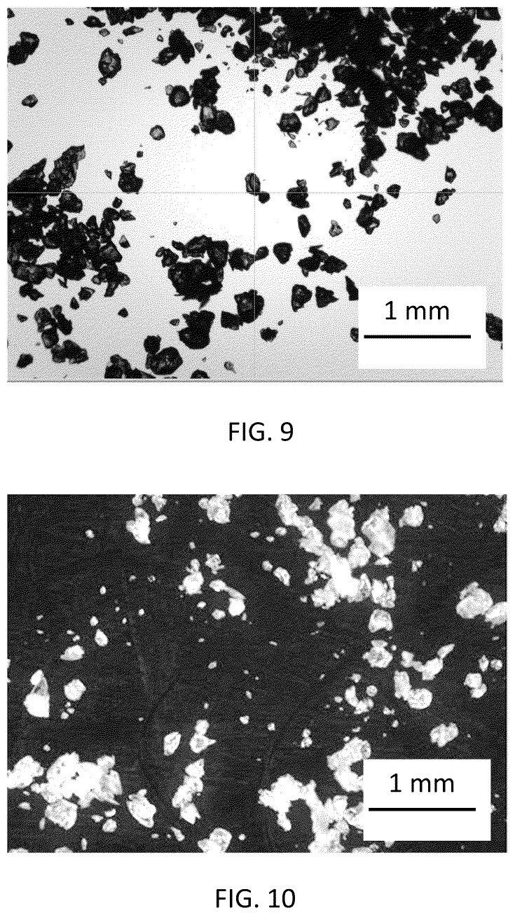

Gelatin/salt sheets having a thickness of about 150 μm were prepared with gelatin G-B. For each sheet to be formed, a 10% (w/w) solution was prepared by dissolving 100 g of gelatin G-B in 900 g of 0.41M aqueous NaCl at 60° C. Approximately 90 g of the gelatin solution was poured into a polypropylene tray (600 cm²). Some gelatin solutions were allowed to gel in the tray then dried at 31° C. with air flow. Other gelatin solutions were not allowed to gel (remained in liquid state) and were dried at 31° C. with air flow. The dried gelatin/salt sheets were thermally conditioned at temperatures of 160° C. or 170° C. and for times ranging from 4 hours to 24 hours. The conditioned gelatin/salt sheets were broken up by hand then ground and sieved to a size range of 63-250 μm. An optical micrograph of example particles is shown in FIG. 6.

Digestion times were determined using a pepsin digestion method or assay as follows. An aqueous solution of pepsin was prepared at a concentration 0.50 mg/ml in 10 mM aqueous HCl. Samples of 20±1 mg of gelatin/salt particles in 20 ml of pepsin solution at 37° C. were prepared in vials and maintained at 37° C. Periodically, each vial was visually assessed by removing it from the bath, swirling the sample, drying the outside of the vial, then holding the vial up to the light to check for a diffraction pattern from suspended particles. The sample was then assigned a digestion score of 0-2 as follows:
0—Not Digested: Diffraction pattern visible in the sample volume
1—Partially Digested: Clearly swollen particles visible
2—Digested: No swollen gelatin particles visible
   Once a digestion score of 2 was found, the time was recorded as the digestion time in hours.

Digestion times are shown in Table 2. The data suggest that digestion times of the particles increase if formed from gelatin/salt sheets dried from a gel state as compared to a liquid state. The data also suggest that digestion times of the particles increase as conditioning temperature of the sheets increases. This increase may be due to increasing crosslink density which is controlled with the thermal conditioning where higher temperature and time resulted in a higher crosslink density.

TABLE 2

| | | Thermal | | | Digestion Time | |
|---|---|---|---|---|---|---|
| | State | Conditioning | | Sheet | | Post Y |
| | Before | Temp. | Time | Thickness | | irradiation |
| Gelatin | Drying | (° C.) | (h) | (μm) | (h)* | (h)** |
| G-A | liquid | 160 | 16 | | 0.5 | 0.4 |
| G-C | liquid | 160 | 16 | | 0.2 | 0.1 |
| G-C | liquid | 180 | 6 | | 2 | 2 |
| G-C | liquid | 180 | 16 | | 0.8 | 0.8 |
| G-C | gel | 140 | 6 | | — | 0.2 |
| G-A | gel | 160 | 16 | | 1 | — |
| G-A | gel | 160 | 16 | | 1 | — |
| G-C | gel | 160 | 16 | | 5 | 7 |
| G-C | gel | 180 | 16 | | 16^{†} | 16^{†} |
| G-B | gel | 160 | 4 | ~150 | 0.6 | — |
| | | 160 | 8 | | 0.9 | — |
| | | 160 | 12 | | 1.4 | — |
| | | 160 | 16 | | 1.7 | — |
| | | 160 | 24 | | 1.9 | — |
| | | 170 | 4 | | 1.4 | — |
| | | 170 | 8 | | 2.2 | — |
| | | 170 | 12 | | 4.3 | — |
| | | 170 | 16 | | 5.0 | — |
| | | 170 | 24 | | 5.3 | — |

*n = 2, repeated once, average shown;
**n = 2;
^{†}estimated time

### Example 3—Digestion Times for Particles Less than 63 μm

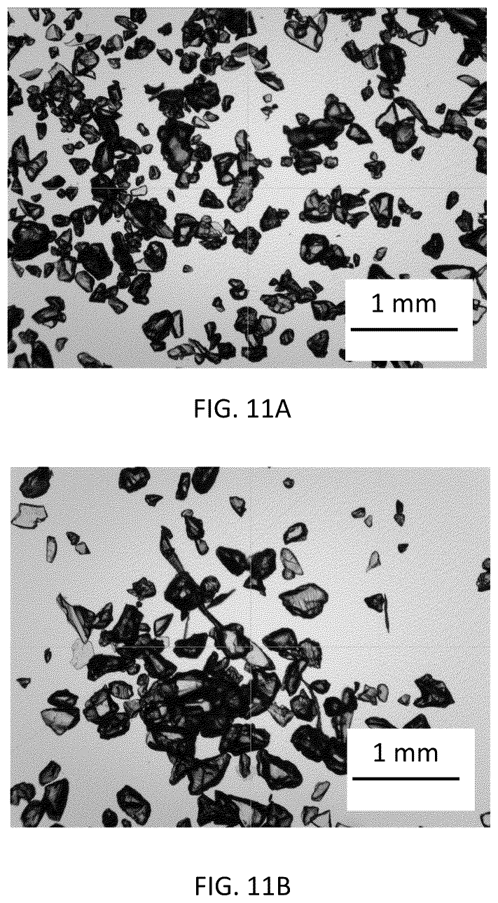

Digestion times were determined for particles sieved to less than 63 μm and obtained from gelatin/salt sheets having a thickness of about 150 μm. A 10% (w/w) solution was prepared by dissolving 100 g of gelatin G-C in 900 g of 0.41M aqueous NaCl at 60° C. Approximately 90 g of the gelatin solution was poured into a polypropylene tray (600 cm²) and allowed to gel. The sheets were thermally conditioned at a temperature of 160° C. and for times ranging from 2 hours to 11 hours. The conditioned sheets were ground and sieved to less than 63 μm. An optical micrograph of example dry particles is shown in FIG. 7.

Digestion times were determined as described above and results are shown in Table 3. The data suggest that smaller particles of the same formulation and conditioning degraded faster than larger equivalent particles implying degradation via erosion rather than bulk degradation. The data also suggest that digestion times of the particles increase as conditioning temperature of the sheets increases. This increase may be due to increasing crosslink density as described for the larger particles.

TABLE 3

| | State | Thermal Conditioning | | Sheet | |
|---|---|---|---|---|---|
| | Before | Temp. | Time | Thickness | Digestion |
| Gelatin | Drying | (° C.) | (h) | (μm) | Time (h) |
| G-C | gel | 160 | 2 | ~150 | 0.15 |
| | | | 5 | | 0.2 |
| | | | 8 | | 0.25 |
| | | | 11 | | 0.3 |

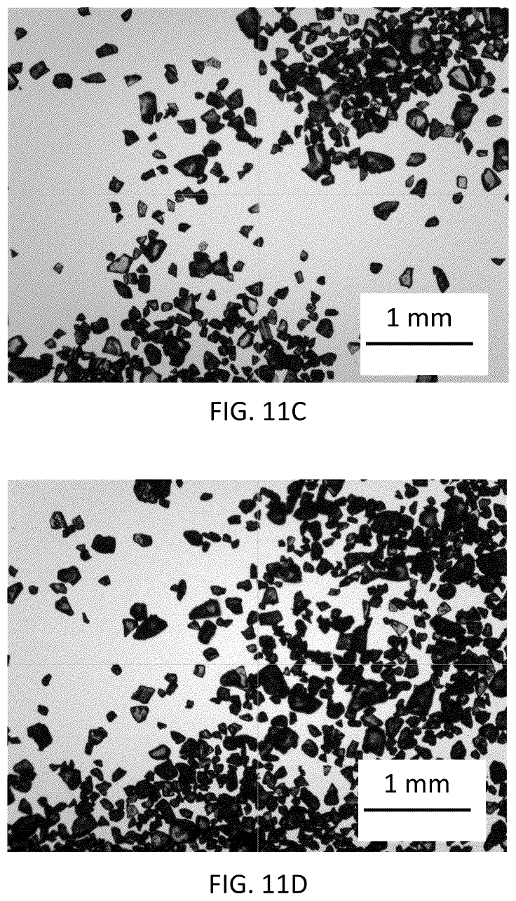

Pepsin digestion was not a suitable method to evaluate a non-crosslinked or lightly crosslinked gelatin/salt particles. For this purpose, phosphate-buffered saline (PBS) at 37° C. was used to dissolve non or lightly thermally conditioned particles. Particles crosslinked for several hours at 160° C. do not dissolve in PBS. FIG. 8 shows digestion times for gelatin/salt particles not thermally conditioned or thermally conditioned at 130° C. or 140° C. for 1 hour as indicated for each sample.

### Example 4—Settling/Separation of Particle Suspensions and Particle Densities

Embozene™ Microspheres, sized 250 μm in diameter, were obtained from Boston Scientific Corporation. These microspheres comprise a polyphosphazene coated trisacryl, acrylamido-PVA and polymethylmethacrylate hydrogel core. Approximately 100 g of the microspheres were suspended in 5 mL of approximately 30% (v/v) contrast agent solution in saline, per the Instructions For Use (IFU).

Approximately 100 mg of gelatin/salt particles sieved to 63-250 μm, prepared as described in Example 2, were suspended in 5 mL solutions of contrast agent solution and saline at volume percentages ranging from 100% (v/v) to 50% (v/v) as shown in Table 4. Approximately 4 mL of each suspension was added to a racked glass culture tube to a depth of about 4.6 cm and allowed to settle. The particles were considered settled when the particles reached the midpoint or 50% of the fluid depth, whether floating or sinking. When settled, the floating particle occupied the top half of the tube, and the sinking particles occupied the lower half of the tube. The “suspended” particles did not reach a settling point in the allotted time. Times to settling and observations are summarized in Table 4.

TABLE 4

| | Vol % | Time to settle | |
|---|---|---|---|
| Suspension | contrast | (min) | Observations |
| Embozene ™ | Per IFU | 8 | Floating |
| | (~30%) | | |
| GS | 100 | 8 | Floating, biphasic |
| GS | 75 | N/A | Suspended at 50 |
| | | | minutes |
| GS | 67 | 11 | Sinking, biphasic |
| GS | 50 | 6 | Sinking, biphasic |

The density of the particles can be calculated based on the volume fraction and density of the gelatin, 82% and 1.3 g/cm³, and NaCl, 18% and 2.17 g/cm³. This results in a calculated density of 1.47 g/cm³. Calculated solution densities of the contrast agent solution/saline mixtures are listed in Table 5. It was assumed that there was no volume change upon mixing. A density of 1.045 g/cm³ was used for saline.

TABLE 5

| Volume Ratio Contrast | Calculated Solution |
|---|---|
| Agent Solution to Saline | Density (g/cm³) |
| 50/50 | 1.20 |
| 67/33 | 1.25 |
| 75/25 | 1.27 |
| 100/0 | 1.349 (from IFU) |

Based on the settling data it can be concluded that the density of the gelatin particles is lower than the calculated density of the gelatin/salt particles, due to the swelling of the particles in an aqueous solution resulting in a swollen density of around 1.27 g/cm³. The swollen density of the smaller particles, <63 μm, matched that of the larger particles 63-250 μm, as to be expected as density is an intrinsic property and crosslinking affects the amount of swelling.

### Example 5—Particle Differences as a Function of Salt

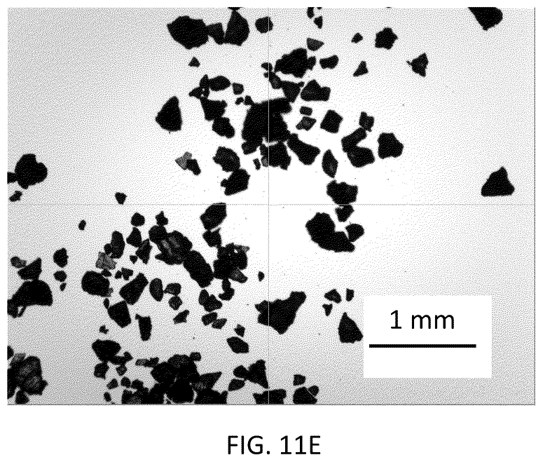

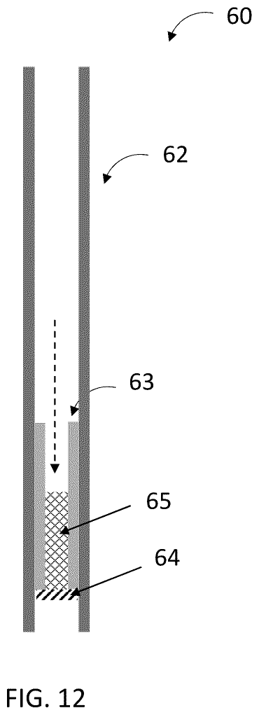

Particles without salt were prepared as follows. Approximately 10 g of gelatin G-B was dissolved in water at 70° C. to give a 10% (w/w) solution of the gelatin. Approximately 90-95 g of the solution was poured into a polypropylene tray (600 cm²) and allowed to gel. The gelled sample was allowed to dry at 31° C. with air flow to form a dried gelatin sheet with an average thickness of about 100 microns. The dried gelatin sheet was then subject to thermal conditioning in an oven at 160° C. for 8 hours. The dried gelatin sheet was ground and sieved to a size range of 63-250 μm. An optical micrograph of example particles is shown in FIG. 9. A second set of particles without salt were prepared and sieved to a size range less than 63 μm; a corresponding optical micrograph is shown in FIG. 10.

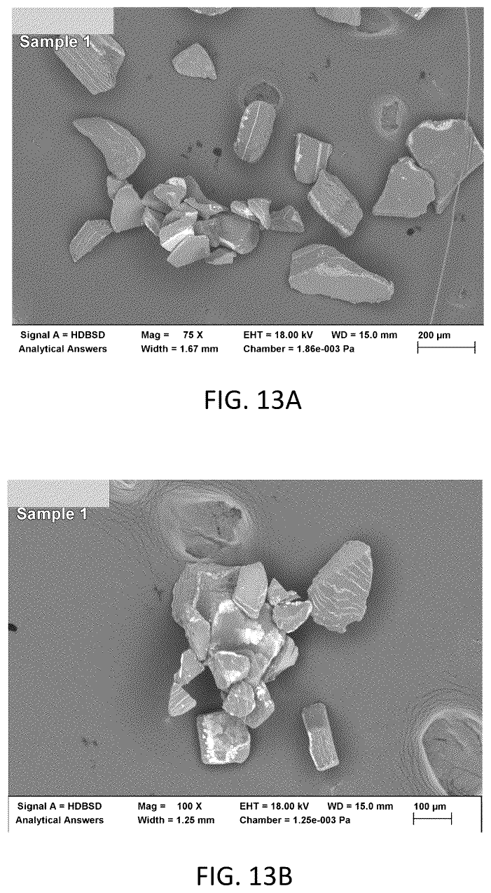

The particles without salt appeared softer and more cohesive with a tendency to agglomerate as compared to particles prepared with salt (described below for FIGS. 11A-11E). FIG. 10 shows the presence of particles less than 63 μm that were not removed by sieving. Generally, sieving was more difficult due to clumping.

A series of particles were similarly prepared as those shown in FIG. 9 except that aqueous NaCl solutions were used instead of water such that the gelatin/salt sheets had an increasing concentration of salt. The NaCl concentrations of the 63-250 μm particles were 5.09 wt %, 9.64 wt %, 17.4 wt %, 29.2 wt % and 44.1 wt %, and optical micrographs are shown in FIGS. 11A-11E, respectively. FIG. 11E shows the presence of very small particles and the particles had a dusty quality.

### Example 6—Packing of Particles

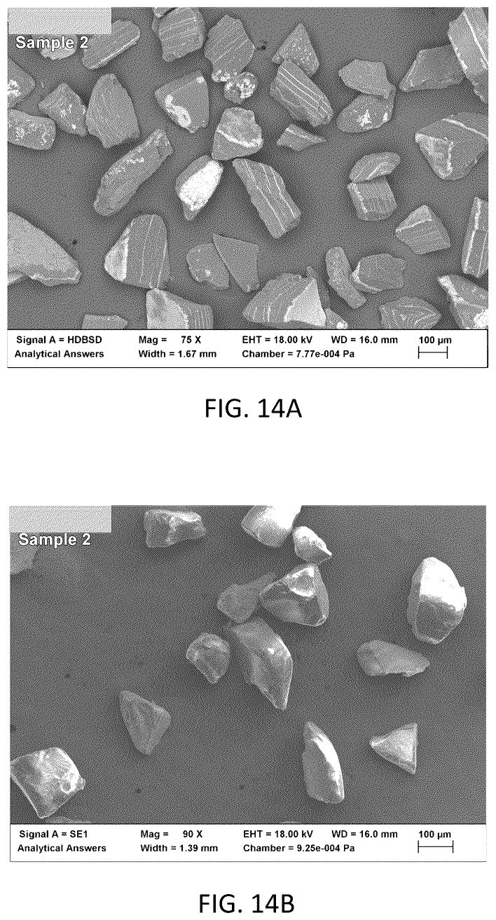

The ability of salted particles to prevent leakage in a vascular vessel was assessed using a testing apparatus. A schematic cross sectional view of testing apparatus 60 is shown in FIG. 12. The apparatus consisted of outer tube 62 having an inner diameter of 0.25 in and fitted with frit 64 having a porosity of 20 μm. Inner tube 63 having an inner diameter of 0.125 in is disposed inside of outer tube 62 and is held in place by frit 64. Water to a height of 45 cm was placed inside testing apparatus 60 and a volume of salted particles were dropped into the water and allowed to settle for 1 minute corresponding to time t=0. Water below the frit was collected and measured after 3 minutes, then again after 4 hours. The direction of flow of water is shown by the dashed arrow.

Results for salted particles sieved to two different size ranges are shown in Table 6. Also included in Table 6 are results for Embozene™ Microspheres, sized 250 μm in diameter, and for a control in which embolic material was not used, and the weight of water was measured after 30 seconds. The salted particles swelled approximately 2.5-6× in size when in suspension.

TABLE 6

| | Embolic | Initial Leaking | Leaking |
|---|---|---|---|
| Embolic | Amount | After 3 Minutes | After 4 Hours |
| Material | (mm³) | (mg) | (mg) |
| None | N/A | 12,000¹ | N/A |
| Embozene | 75 | 5210 | 4300 |
| Sieved to less than 63 μm | 20 | 60 | 0 |
| Sieved to 63-250 μm | 90 | 80 | 0 |

¹After 30 seconds

### Example 7—Scanning Electron Microscopy of Gelatin/Salt Particles

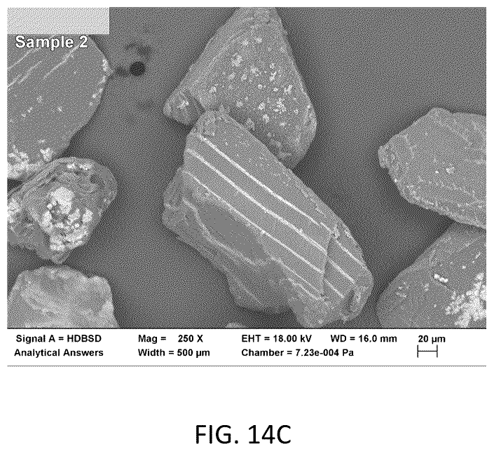

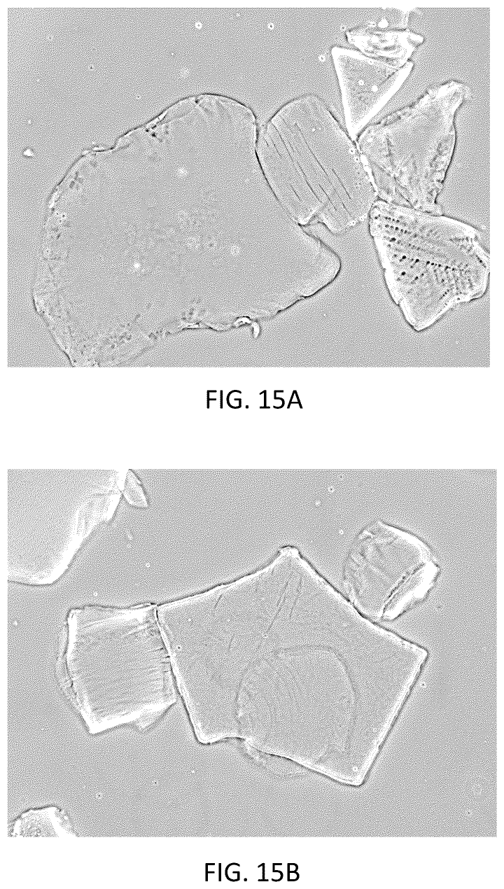

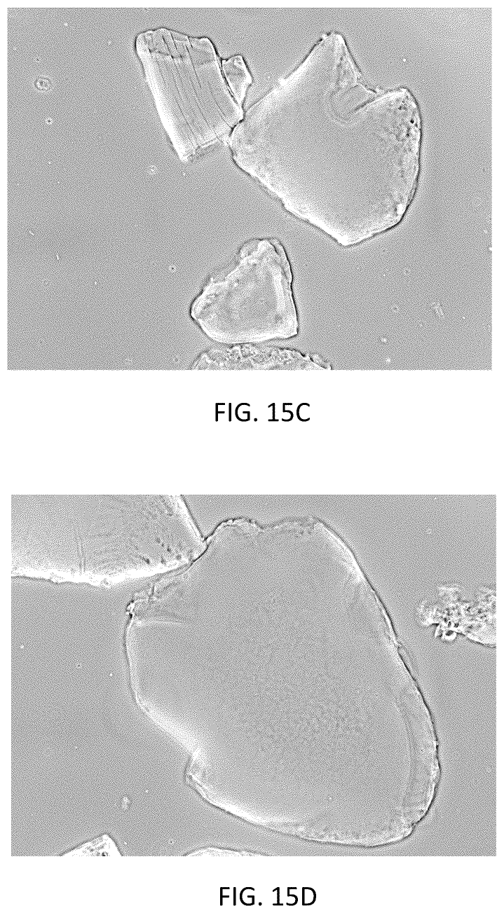

Gelatin/salt particles sieved to a size range of 63-250 μm were prepared as described for gelatin G-B of Example 2. Images of two samples of dry particles and one sample of hydrated particles were obtained using scanning electron microscopy (SEM). FIGS. 13A and 13B are images at 75× and 100× magnifications, respectively, of one sample of dry gelatin/salt particles. FIGS. 14A-14C are images at 75×, 90×, and 250× magnifications, respectively, of a second sample of dry gelatin/salt particles. FIGS. 15A-15D are images at 100× magnification of hydrated gelatin/salt particles.

### Example 8—Experiments with Gelatin Foam

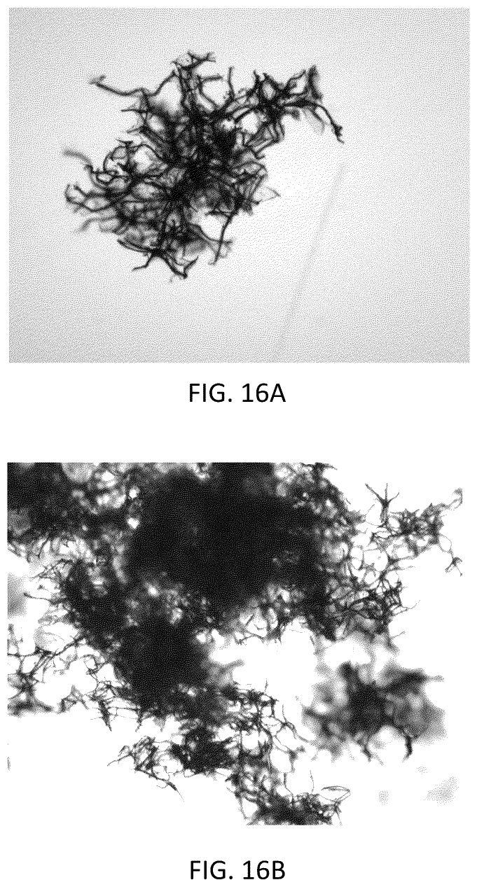

Gelatin foam sheets (gelatin from porcine skin) were ground as described above to give shredded flakes and filaments which seemed fluffy. The sheets were received slightly crosslinked (method unknown) and the sheet was not further thermally conditioned. The shredded gelatin foam was not sievable due to static electricity. Optical micrographs are shown in FIGS. 16A and 16B. The micrographs show aggregated shredded and ground foam. Approximately 100 mg of the shredded gelatin foam was suspended in 5 mL of the saline/contrast agent solution. Embolization was evaluated and found to be successful.

### Example 9—Experiments with Salted Gelatin Foam

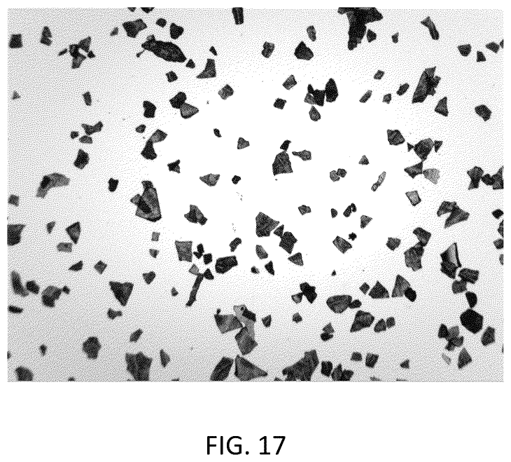

Approximately 90 mL of 1M NaCl (aq) was added to approximately 3 g of gelatin sponge. The salt solution was added to the sponge and to force hydration, the sponge was repeatedly compressed and allowed to relax. After the voids in the foam were filled with the aqueous salt solution, the sample was dried at 50° C. The dried sample was ground and sieved to a size range of 63-250 μm. Unlike the unsalted ground gelatin foam, the ground particles from the salted gelatin sponge could be sieved. An optical micrograph of the salted gelatin sponge particles is shown in FIG. 17. Approximately 100 mg of the gelatin particles were suspended in 5 mL of the saline/contrast agent solution. Embolization was evaluated and found to be successful.

### Further Inventive Concepts

A1. A collection of dry gelatin particles comprising crosslinked gelatin and from about 1 wt % to about 66 wt % metal halide salt, having a sieve particle size of no more than 2000 microns.
A2. The collection of dry gelatin particles of inventive concept A1 wherein the crosslinked gelatin is formed with a gelatin having an uncrosslinked bloom value from about 50 to about 325.
A3. The collection of dry gelatin particles of inventive concept A1 having from about 1.5 wt % to about 55 wt % sodium chloride, calcium chloride or mixture thereof.
A4. The collection of dry gelatin particles of inventive concept A1 having from about 2 wt % to about 40 wt % sodium chloride, calcium chloride or mixture thereof.
A5. The collection of dry gelatin particles of inventive concept A1 wherein a majority of the gelatin particles by weight have a non-spheroid shape with an aspect ratio of at least about 3.
A6. The collection of dry gelatin particles of inventive concept A4 wherein a majority of the gelatin particles by weight have platelet shapes.
A7. The collection of dry gelatin particles of inventive concept A1 wherein the gelatin particles have a sieve particle size distribution of from about 20 microns to about 1000 microns.
A8. The collection of dry gelatin particles of inventive concept A1 wherein the gelatin particles have a sieve particle size distribution of from about 63 microns to about 250 microns.
A9. The collection of dry gelatin particles of inventive concept A1 wherein the sieve particle size is no more than about 63 microns.
A10. The collection of dry gelatin particles of inventive concept A1 wherein the gelatin particles, upon hydration, have a swelled density providing suspension in a 50:50 by weight blend of saline and contrast agent having a density of about 1.20 g/mL.
A11. The collection of dry gelatin particles of inventive concept A1 wherein the gelatin particles have an in vitro pepsin digestion time of from about 2 minutes to about 48 hours.
A12. The collection of dry gelatin particles of inventive concept A1 wherein the gelatin particles have an in vitro pepsin digestion time of from 5 minutes to about 24 hours.
A13. The collection of gelatin particles of inventive concept A1 wherein the gelatin particles have an in vivo recanalization rate of from about 30 minutes to about 60 days.
A14. The collection of gelatin particles of inventive concept A1 wherein the gelatin particles are formed by grinding a gelatin sheet that has been thermally crosslinked at from about 100° C. to about 200° C. for from about 5 minutes to about 48 hours.
A15. The collection of gelatin particles of inventive concept A1 wherein a majority of the gelatin particles by weight have a platelet shape and the platelet shaped particles have an average thickness from about 25 microns to about 200 microns, wherein the average thickness of a collection averages across the expanse of each platelet and averages over all of the platelets of the collection.
B1. A method for forming biocompatible gelatin particles, the method comprising:
   grinding a sheet of gelatin to form gelatin particles, wherein the sheet of gelatin comprises crosslinked gelatin with from about 1 wt % to about 66 wt % metal halide salt; and
   sieving the gelatin particles to obtain a desired distribution of particles with a maximum particle size no more than about 2000 microns.
B2. The method of inventive concept B1 wherein the sheet of gelatin is formed by drying a layer of gelatin solution or gel in a tray wherein the gelatin is salted and has an average dry thickness from about 20 microns to about 1000 microns.
B3. The method of inventive concept B2 wherein the layer of gelatin solution or gel is a gel and wherein the drying is performed at a temperature below the melting point of the gelatin and wherein the sheet of gelatin is formed by pouring a warm solution of dissolved gelatin and salt into a tray.
B4. The method of inventive concept B2 wherein the layer of gelatin solution or gel is a solution and wherein the drying is performed at a temperature above the melting point of the gelatin gel and wherein the sheet of gelatin is formed by pouring a warm solution of dissolved gelatin and salt into a tray.
B5. The method of inventive concept B1 further comprising drying a gel comprising salted gelatin to form the sheet of gelatin, wherein the drying is performed at room temperature by blowing gas across the surface.
B6. The method of inventive concept B1 further comprising drying an aqueous solution comprising salted gelatin to form the sheet of gelatin, wherein the drying is performed by blowing heated gas across the surface.
B7. The method of inventive concept B1 wherein the sheet of gelatin is formed by thermally crosslinking a dried layer comprising gelatin and salt.
B8. The method of inventive concept B7 wherein the dried layer has an average thickness from about 20 microns to about 1000 microns.
B9. The method of inventive concept B7 wherein the dried layer has an average thickness from about 30 microns to about 200 microns.
B10. The method of inventive concept B1 further comprising forming the sheet of gelatin, wherein forming the sheet of gelatin comprises thermal crosslinking and wherein the thermal crosslinking is performed at a temperature from about 100° C. to about 200° C. for from about 5 minutes to about 48 hours.
B11. The method of inventive concept B1 wherein the sheet of gelatin is formed by drying gelatin foam imbibed with an aqueous salt solution.
B12. The method of inventive concept B1 wherein the metal halide salt comprises sodium chloride, calcium chloride or a mixture thereof.
B13. The method of inventive concept B1 wherein the grinding comprises milling the sheet or fragments of the sheet to form milled gelatin particles.
B14. The method of inventive concept B1 wherein the sieving comprises passing the gelatin particles after grinding through a mechanical sieve that vibrates the gelatin particles over a selected sieve mesh.
B15. The method of inventive concept B1 wherein a majority of the gelatin particles by weight have a platelet shape.
B16. The method of inventive concept B15 wherein the platelet shaped gelatin particles have an average thickness from about 25 microns to about 200 microns, wherein the average thickness of a collection averages across the expanse of each platelet and averages over all of the platelets of the collection.
C1. A method for making a sheet of salted thermally crosslinked gelatin, the method comprising:
   heating a sheet of dried salted gelatin at a temperature from 100° C. to about 200° C. for from about 5 minutes to about 48 hours, wherein the sheet of dried salted gelatin comprises gelatin and from about 1 wt % to about 66 wt % salt to form a sheet of thermally crosslinked gelatin.
C2. The method of inventive concept C1 wherein the dry salted gelatin has an initial dry average thickness from about 20 microns to about 1000 microns
C3. The method of inventive concept C1 wherein the heating is performed at a temperature from 120° C. to about 180° C. for from about 20 minutes to about 16 hours.
C4. The method of inventive concept C1 wherein the dry salted gelatin comprises from about 1.5 wt % salt and about 50 wt % salt and has an initial dry average thickness from about 25 microns to about 300 microns.
C5. The method of inventive concept C1 further comprising:
   forming the sheet of dry salted gelatin by soaking a foamed gelatin sheet in salted water and drying the soaked foamed gelatin sheet.
C6. The method of inventive concept C1 further comprising:
   dissolving gelatin to form an aqueous solution comprising salt; and
   drying the solution of water, salt and dissolved gelatin in a tray to form the dried salted gelatin sheet.
C7. The method of inventive concept C6 wherein the solution cools to form a gel and drying is performed at a temperature that does not melt the gel.
C8. The method of inventive concept C6 wherein the dried salted gelatin sheet has an average thickness from about 20 microns to about 500 microns.
C9. The method of inventive concept C1 further comprising grinding the sheet of salted gelatin or fragments thereof to form particles of salted gelatin.
C10. The method of inventive concept C9 wherein the grinding is performed in a mill at a milling rate from 1000 rpm to about 30,000 rpm for a time from about 15 seconds to about 40 minutes.
C11. The method of inventive concept C9 further comprising sieving the particles of salted gelatin to obtain sieved particles having a target particle size distribution.
C12. The method of inventive concept C9 wherein a majority of the particles by weight have a platelet shape.
C13. The method of inventive concept C12 wherein the platelet shaped gelatin particles have an average thickness from about 25 microns to about 200 microns, wherein the average thickness of a collection averages across the expanse of each platelet and averages over all of the platelets of the collection.
C14. The method of inventive concept C11 wherein the gelatin particles have a sieve particle size distribution of from about 63 microns to about 250 microns.
C15. The method of inventive concept C11 wherein the sieve particle size is no more than about 63 microns.
C16. The method of inventive concept C11 wherein the sieved particles have an in vitro pepsin digestion time from about 5 minutes to about 24 hours.
D1. A method for performing temporary occlusion of a blood vessel, the method comprising:
   injecting hydrated crosslinked gelatin particles into the parent vessel of target vasculature for occlusion, wherein a majority of the hydrated crosslinked gelatin particles by weight having ellipsoidal shape with an aspect ratio of at least about 3, wherein the dry particles prior to hydration have a sieved size of no more than 2000 microns and wherein the target vessel completely occludes and recanalization of the vessel occurs in a period of time from about 30 minutes to about 60 days.
D2. The method of inventive concept D1 wherein the target vasculature comprises fine vasculature associated with inflammation accompanying neovascularization.
D3. The method of inventive concept D2 wherein the neovascularization is related to osteoarthritis.
D4. The method of inventive concept D1 wherein for injecting, the crosslinked gelatin particles are dispersed in a solution.
D5. The method of inventive concept D4 wherein the solution comprises contrast agent and aqueous saline.
D6. The method of inventive concept D5 wherein the solution comprises contrast agent and roughly 50 vol % saline.
D7. The method of inventive concept D4 wherein the solution comprises from about 10 mg to about 400 mg crosslinked gelatin particles.
D8. The method of inventive concept D7 wherein the solution has a volume from about 0.5 ml to about 10 ml.
D9. The method of inventive concept D4 wherein the solution comprises from about 1 mg/ml to about 100 mg/ml crosslinked gelatin particles.
D10. The method of inventive concept D1 wherein the crosslinked gelatin particles are thermally crosslinked.
D11. The method of inventive concept D10 wherein the thermal crosslinking is performed at a temperature from 100° C. to about 200° C. for from about 5 minutes to about 48 hours.
D12. The method of inventive concept D1 wherein a majority of the particles by weight in dry form prior to hydration have a platelet shape.
D13. The method of inventive concept D12 wherein the platelet shaped gelatin particles have an average thickness from about 25 microns to about 200 microns, wherein the average thickness of a collection averages across the expanse of each platelet and averages over all of the platelets of the collection.
D14. The method of inventive concept D1 wherein the gelatin particles comprise from about 1.5 wt % to about 50 wt % salt.
D15. The method of inventive concept D1 wherein the gelatin particles in dry form prior to hydration have a sieve particle size distribution of from about 63 microns to about 250 microns.
D16. The method of inventive concept D1 wherein the sieve particle size in dry form prior to hydration is no more than about 63 microns.
D17. The method of inventive concept D1 wherein the crosslinked gelatin particles have an in vitro pepsin digestion time from about 5 minutes to about 24 hours.
D18. The method of inventive concept D1 wherein the vessel undergoes recanalization in from about 10 minutes to about 60 days.
E1. A medical system for delivery of embolic material, the medical system comprising:
   a catheter comprising a hub and a tubular element, the catheter being suitable for delivery through a patient's vasculature to reach a target vessel or the vicinity thereof;
   a delivery component comprising a reservoir, wherein the delivery component comprises a connector and an engagement element to deliver material from the delivery component through the connector, the connector being suitable to connect to the hub of the catheter to form a sealed engagement in a configuration
   configured to deliver the embolic material through the catheter and out an end of the tubular element; and
   a collection of non-spheroidal, crosslinked gelatin particles having a dry sieve particle size of no more than 2000 microns.
E2. The medical system of inventive concept E1 further comprising a suspension fluid, wherein the non-spheroidal crosslinked gelatin particles can be suspended in the fluid to form suspended embolic material within the reservoir, wherein the delivery component comprises a syringe, and wherein the reservoir is the lumen of the syringe configured such that depressing a plunger of the syringe delivers the suspended embolic material through the catheter when the delivery component is connected to the hub.
E3. The medical system of inventive concept E2 wherein the collection of non-spheroidal, crosslinked gelatin particles have a weight from about 10 mg to about 400 mg.
E4. The medical system of inventive concept E3 wherein the suspension fluid has a volume from about 0.5 ml to about 10 ml.
E5. The medical system of inventive concept E1 wherein a majority of the dry gelatin particles by weight have a platelet shape.
E6. The medical system of inventive concept E5 wherein the platelet shaped, dry gelatin particles have an average thickness from about 25 microns to about 200 microns and an average aspect ratio of at least about 2, wherein the average thickness of a collection averages across the expanse of each platelet and averages over all of the platelets of the collection.
E7. The medical system of inventive concept E5 wherein the dry gelatin particles have a sieve particle size distribution of from about 63 microns to about 250 microns.
E8. The medical system of inventive concept E5 wherein the dry gelatin particles have a sieve particle size is no more than about 63 microns.
E9. The medical system of inventive concept E1 wherein a majority by weight of the hydrated particles have an ellipsoidal shape.
E10. The medical system of inventive concept E9 wherein a majority of the hydrated particles by weight have an aspect ratio of at least about 2.
E11. The medical system of inventive concept E1 wherein the dry gelatin particles have from about 1 wt % to about 66 wt % salt.
E12. The medical system of inventive concept E1 wherein the dry gelatin particles have from about 1.5 wt % to about 40 wt % salt.
E13. The medical system of inventive concept E1 wherein the dry gelatin particles are formed by milling a sheet of salted gelatin formed by drying a gel.
E14. The medical system of inventive concept E1 wherein the dry gelatin particles are formed by milling a sheet of salted gelatin formed by drying a salted aqueous gelatin melt.
E15. The medical system of inventive concept E1 wherein the dry gelatin particles are thermally crosslinked.
E16. The medical system of inventive concept E1 wherein the dry gelatin particles are radiation crosslinked.
E17. The medical system of inventive concept E1 further comprising a biocompatible suspension fluid, wherein the biocompatible suspension fluid comprises a blend of contrast agent and aqueous saline.
E18. The medical system of inventive concept E13 further comprising a biocompatible suspension fluid, wherein the biocompatible suspension fluid comprises contrast agent and roughly 50 vol % saline.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to measurement error for the particular parameter unless explicitly indicated otherwise.

What is claimed is:

1. A collection of dry gelatin particles comprising crosslinked gelatin having a sieve particle size of no more than 2000 microns and a majority of the particles by weight having non-spheroid shape with an aspect ratio of at least about 2.

2. The collection of dry gelatin particles of claim 1 wherein a majority by weight of the dry gelatin particles comprise platelets.

3. The collection of dry gelatin particles of claim 2 wherein the platelets have an average thickness from about 20 microns to about 1000 microns, wherein the average thickness of a collection averages across the expanse of each platelet and averages over all of the platelets of the collection.

4. The collection of dry gelatin particles of claim 2 wherein the platelets have an average thickness from about 25 microns to about 200 microns, wherein the average thickness of a collection averages across the expanse of each platelet and averages over all of the platelets of the collection.

5. The collection of dry gelatin particles of claim 2 wherein the platelets have a thickness variation no more than about ±50 microns.

6. The collection of gelatin particles of claim 2 wherein the platelets comprise a plate, and a perimeter of the plate is irregular with respect to individual platelets and between platelets of the collection.

7. The collection of dry gelatin particles of claim 1 having a sieve particle size from 63 microns to 250 microns.

8. The collection of dry gelatin particles of claim 1 having a sieve particle size of less than 63 microns.

9. The collection of dry gelatin particles of claim 1 wherein the crosslinked gelatin comprises thermally crosslinked gelatin.

10. The collection of dry gelatin particles of claim 9 wherein the thermally crosslinked gelatin was dried from a gelled form.

11. The collection of dry gelatin particles of claim 9 wherein the thermally crosslinked gelatin was dried from a melted state.

12. The collection of dry gelatin particles of claim 9 wherein the thermally crosslinked gelatin is formed by thermally crosslinking a gelatin at a temperature from about 100° C. to about 200° C. for from about 5 minutes to about 48 hours.

13. The collection of dry gelatin particles of claim 1 wherein the crosslinked gelatin is formed from a gelatin having an uncrosslinked bloom value from about 50 to about 325.

14. The collection of dry gelatin particles of claim 1 comprising from about 1 wt % to about 66 wt % metal halide salt.

15. The collection of dry gelatin particles of claim 1 comprising from about 2 wt % to about 40 wt % sodium chloride, calcium chloride or mixture thereof.

16. The collection of dry gelatin particles of claim 1 wherein upon hydration the dry gelatin particles form hydrated gelatin particles having an in vitro pepsin digestion time of from 5 minutes to about 24 hours.

17. The collection of dry gelatin particles of claim 1 wherein upon hydration the dry gelatin particles form hydrated gelatin particles having an in vivo recanalization rate of from about 30 minutes to about 60 days.

18. The collection of dry gelatin particles of claim 1 wherein a majority of the particles by weight having non-spheroid shape with an aspect ratio of at least about 2.
